(19) United States
(12) Patent Application Publication
(10) Pub. No.: US 2013/0331537 A1
(43) Pub. Date: Dec. 12, 2013

(54) POLYETHER URETHANE AND POLYETHER UREA BASED COPOLYMERS AND METHODS RELATED THERETO

(71) Applicant: POLY-MED, INC., Anderson, SC (US)

(72) Inventors: Kenneth David Gray, JR., Clemson, SC (US); Joel Thomas Corbett, Anderson, SC (US); Georgios Theofanis Hilas, Anderson, SC (US)

(21) Appl. No.: 13/913,217

(22) Filed: Jun. 7, 2013

Related U.S. Application Data
(60) Provisional application No. 61/657,578, filed on Jun. 8, 2012.

(51) Int. Cl. A61L 27/18

(52) U.S. Cl. CPC A61L 27/18
USPC 528/61

(57) ABSTRACT

Polymeric materials referred to herein as PEU include a plurality of linking groups selected from urea and urethane groups, and a plurality of segments located between adjacent linking groups, where at least some of the segments of the PEU are polyoxyalkylene, but other exemplary segments include hydrocarbons, polyesters, polycarbonates and polysiloxanes. The polymeric materials are biocompatible and may be used, for example, to replace or supplement cartilage in an articulated joint.

### CROSS-REFERENCE TO RELATED APPLICATION

This application claims the benefit under 35 U.S.C. §119(e) of U.S. Provisional Patent Application No. 61/657,578 filed Jun. 8, 2012, which application is incorporated herein by reference in its entirety.

### FIELD OF THE INVENTION

The present invention relates generally to polymeric materials, in particular to biomaterials suitable for implantation into a mammal or other animal, and methods of manufacturing the biomaterial and various uses thereof.

### BACKGROUND

Biocompatible polymeric materials are in increasing demand, particularly biomaterials that can be used to supplement or replace natural materials within a person that degrade upon aging, or need to be replaced upon injury. Within a person, the natural materials tend to be maintained by the body and thus are dynamic materials. A challenge with finding a synthetic bioequivalent is that the synthetic material is not nourished or physiologically supported by the host, and thus the bioequivalent is preferably inherently stable over a long period of time. Another challenge is that many implants are placed in stressful environments, i.e., environments that are under repeated mechanical stress, or are constantly exposed to biological fluids that can degrade the polymers. The polymeric materials must therefore be particularly durable. In some environments, it is necessary that the implant be able to absorb moisture from the surrounding biological fluids, which normally will tend to soften the polymer and make it less resistant to mechanical stress.

There is a need for biocompatible polymeric materials that can absorb moisture but still be durable upon exposure to repeated mechanical stress. The present invention is directed towards meeting this need.

### SUMMARY

In one aspect there is provided a polymer comprising repeating oxyalkylene groups (i.e., a polyether) and repeating linkages selected from urethane and urea. Such polymers are referred to herein as PEU, where exemplary PEUs include polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxne urethane (PESUT), and polyether siloxane urethane urea (PESUU).

In another aspect, there is provided a method of manufacturing a PEU. The method includes reacting an aliphatic or aromatic diisocyanate with a polyol or polyamine. Either of the polyol or polyamine may have two or more reactive groups. A diol or diamine can be used to prepare a linear polymer, and a polyol or polyamine with greater than two reactive groups can be used to prepare a crosslinked PEU. Either of the polyol or polyamine may (or may not) incorporate additional functionality, e.g., carbonate or ester or siloxane functionality. The product of this reaction may optionally be “chain extended” by reaction with additional polyol or polyamine. A diol or diamine may be used to produce linear PEUs, and a polyol or polyamine with more than two reactive groups can be used to produce crosslinked PEUs.

In one embodiment, the polymer compositions of the present disclosure are prepared entirely from reactants having two reactive groups, while in another embodiment the polymer compositions of the present disclosure are prepared from reactants having two and more than two (poly-reactive, e.g., polyol or polyamine) reactive groups. In other words, in one optional embodiment, any of the PEUs described herein may be prepared from reactants including some amount of tri- or higher reactive groups, in order to introduce a degree crosslinking into the polymer composition. Thus, in one embodiment the polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), or polyether siloxane urethane urea (PESUU) does not incorporate cross-linking, however in another embodiment the polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), and polyether siloxane urethane urea (PESUU) includes some degree of crosslinking.

The polyol or polyamine used for the chain extension may (or may not) incorporate additional functionality, e.g., carbonate or ester functionality. The PEU will, however, contain some polyether functionality, and may optionally contain a plurality of carbonate or ester or siloxane groups in addition to a plurality of urethane and/or urea groups.

In one embodiment the present disclosure provides a polymer composition which is the reaction product of reactants comprising or consisting of a diisocyanate, a diamine and a polyetherdiol, where the diisocyanate is used to form a pre-polymer by reaction with either the diamine or the diol, and then the pre-polymer is used to form the polymer by reaction with the reactant not used to form the pre-polymer, i.e., if the pre-polymer was formed by reaction of diisocyanate and diamine, then the polymer is formed by reaction of pre-polymer and diol, while if the pre-polymer was formed by reaction of diisocyanate and diol, then the polymer is formed by reaction of the pre-polymer and diamine. The term “a” refers to one or more, e.g., a single structure and a blend of different structures.

Accordingly, in one aspect the present disclosure provides a polymer composition which is the reaction product of a pre-polymer and a diamine, where the pre-polymer is the reaction product of a diisocyanate and a polyetherdiol. Optionally, any one or more of the following may be used to further describe this polymer composition and its preparation: the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyetherdiol is a block copolymer of two or more oxyalkylene sequences where this block copolymer may be used as the sole polyetherdiol reactant or it may be used in combination with a different polyetherdiol reactant (e.g., a homopolymeric polyetherdiol formed from oxyalkylene sequences selected from the group consisting of oxyethylene, oxypropylene, oxytrimethylene and oxytetramethylene sequences) to provide a blend which is the polyetherdiol component of the reactants; the diamine is an aliphatic diamine; the diisocyanate is an aliphatic diisocyanate; the polymer is bio-stable; the polymer absorbs at least 50% of its weight in water when immersed in 1% aqueous methyl cellulose at 37° C. for 16 hours; the polymer has a COF of 0.001 to 0.15; the polymer has an intrinsic viscosity of 3-8 dl/g. A polymer chain may be described in terms of its structural components rather than in terms of the reactants by which it may be formed. In the present case the polymer chain is a polyurea, having a plurality of urea groups separated alternately by aliphatic groups (contributed by the aliphatic diamine) and polymeric blocks (contributed by the pre-polymer). In other words, the structure may be described by repeating -[urea-aliphatic-urea-polymer block]-units. The polymer block is a polyurethane, having a plurality of urethane (also known as carbamate) groups separated alternatively by aliphatic groups (contributed by the diisocyanate) and polyether groups. In other words, the structure of the polymer block may be described by repeating -[urethane-aliphatic-urethane-polyether]- units. The polyether segments may optionally be selected from oxyethylene, oxypropylene, oxytrimethylene and oxytetramethylene, and in one embodiment the polymer chain contains more than one of these polyether segments, for example, the polymer contains oxyethylene, oxypropylene and oxytetramethylene groups, where optionally the oxyethylene and oxypropylene are arranged in a block copolymer arrangement (e.g., oxyethylene block-oxypropylene block-oxyethylene block). The polymer block may also be referred to as a polyether polyurethane, and the polymer itself may be referred to as a poly ether urethane urea. In one embodiment, the polymer is bio-stable, absorbs at least 50% of its weight in water when immersed in 1% aqueous methyl cellulose at 37° C. for 16 hours, has a COF of 0.001 to 0.15, and has an intrinsic viscosity of 3-8 dl/g.

In a related aspect, the present disclosure provides a polymer composition which is the reaction product of a pre-polymer and a polyetherdiol, where the pre-polymer is the reaction product of a diisocyanate and a diamine. Optionally, any one or more of the following may be used to further describe this polymer composition and its preparation: the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyetherdiol is a block copolymer of two or more oxyalkylene sequences where this block copolymer may be used as the sole polyetherdiol reactant or it may be used in combination with a different polyetherdiol reactant (e.g., a homopolymeric polyetherdiol formed from oxyalkylene sequences selected from the group consisting of oxyethylene, oxypropylene, oxytrimethylene and oxytetramethylene sequences) to provide a blend which is the polyetherdiol component of the reactants; the diamine is an aliphatic diamine; the diisocyanate is an aliphatic diisocyanate; the polymer is bio-stable; the polymer absorbs at least 50% of its weight in water when immersed in 1% aqueous methyl cellulose at 37° C. for 16 hours; the polymer has a COF of 0.001 to 0.15; the polymer has an intrinsic viscosity of 3-8 dl/g. A polymer chain may be described in terms of its structural components rather than in terms of the reactants by which it may be formed. In the present case the polymer chain is a polyurethane, having a plurality of urethane groups separated alternately by polyether groups (contributed by the polyether diol) and polymeric blocks (contributed by the pre-polymer). In other words, the structure may be described by repeating -[urethane-polyether-urethane-polymer block]- units. The polymer block is a polyurea, having a plurality of urea groups separated alternatively by first aliphatic groups (contributed by the diisocyanate) and second aliphatic groups (contributed by the diamine). In other words, the structure of the polymer block may be described by repeating -[urea-first aliphatic-urea-second aliphatic]- units. The polyether segments may optionally be selected from oxyethylene, oxypropylene, oxytrimethylene and oxytetramethylene, and in one embodiment the polymer chain contains more than one of these polyether segments, for example, the polymer contains oxyethylene, oxypropylene and oxytetramethylene groups, where optionally the oxyethylene and oxypropylene are arranged in a block copolymer arrangement (e.g., oxyethylene block-oxypropylene block-oxyethylene block). The polymer block may also be referred to as a polyurea, and the polymer itself may be referred to as a poly ether urethane urea. In one embodiment, the polymer is bio-stable, absorbs at least 50% of its weight in water when immersed in 1% aqueous methyl cellulose at 37° C. for 16 hours, has a COF of 0.001 to 0.15, and has an intrinsic viscosity of 3-8 dl/g.

The details of one or more embodiments and aspects are set forth in the description below. Other features, objects and advantages will be apparent from the description and the claims. In addition, the disclosures of all patents and patent applications referenced herein are incorporated by reference in their entirety.

### DETAILED DESCRIPTION OF THE INVENTION

### PEU General Chemical Description

The present invention provides polymeric materials referred to herein as PEU, and methods related thereto. The PEU is a polymeric material that includes a plurality of linking groups selected from urea and urethane groups. The PEU will additionally include a plurality of segments located between adjacent linking groups. In other words, the PEU may be described in whole or part as having portions described as urethane-segment-urethane and/or urea-segment-urea and/or urea-segment-urethane which may also be written as urethane-segment-urea. At least some of the segments of the PEU are polyoxyalkylene. Other exemplary segments include hydrocarbons, polyesters, polycarbonates and polysiloxanes. The segments that do not comprise entirely hydrocarbon will comprise some hydrocarbon located between adjacent functional groups. For example, hydrocarbon will be located between adjacent ester groups of a polyester, between adjacent ether groups of a polyether, between adjacent siloxane groups of a polysiloxane, and between adjacent carbonate groups of a polycarbonate.

The polyoxyalkylene segments have alkylene groups between adjacent oxygen atoms. The polyoxyalkylene segments will contain at least one type of oxyalkylene sequence selected from the group consisting of oxyethylene, oxypropylene, oxytrimethylene, and oxytetramethylene sequences.

A segment may contain entirely hydrocarbon, and the hydrocarbon may be aliphatic or aromatic. In the case of an aliphatic hydrocarbon, the number of carbon atoms in the hydrocarbon may vary between 1 and about 12. When the hydrocarbon is an aromatic hydrocarbon, it will contain at least 6 carbons and may contain as many as about 16 carbons. In various specific aspects, the hydrocarbon contains 2 or 3 or 4 or 5 or 6 or 7 or 8 or 9 or 10 carbon atoms. When hydrocarbon is between two functional groups, e.g., between two ester or two carbonate groups, in various aspects the hydrocarbon as 2 or 3 or 4 or 5 or 6 or more carbons. The hydrocarbon may be saturated.

Exemplary PEUs include polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), and polyether siloxane urethane urea (PESUU).

### Reactants

The PEU may be prepared by reacting together various multi-functional (preferably di-functional) reactants to form linking groups, the linking groups being formed from a functional group of a first reactant reacting with a functional group of a second reactant.

A diisocyanate may be used as a reactant to form the PEU. As is well known, the reaction between a hydroxyl (alcohol) group and an isocyanate group will provide a urethane group, while the reaction between an amine group and an isocyanate group will provide a urea group. Exemplary aliphatic diisocyanates include tetramethylene diisocyanate, hexamethylene diisocyanate, octamethylene diisocyanate, decamethylene diisocyanate, dodecamethylene diisocyanate, and cyclohexane bis-(methylene isocyanate). Aromatic diisocyanates may additionally, or alternatively, be used as a reactant to form the PEU.

A polyether diol may be used as a reactant to form the PEU. The polyether diol will introduce polyoxyalkylene segments, in other words polyether segments, into the PEU. The polyether diol may comprise a homopolymer of oxyalkylene groups, or a copolymer of two different oxyalkylene groups. The copolymer may be a random or block copolymer, for example, a diblock copolymer, or a triblock copolymer. Exemplary oxyalkylene moieties include oxyethylene, oxypropylene, oxytrimethylene, and oxytetramethylene.

A polyether diamine may be used as a reactant to form the PEU. When a polyether diamine is reacted with a diisocyanate-containing reactant, the result will be a polyether urea moiety. The polyether diamine may comprise a homopolymer of oxyalkylene groups, or a copolymer of two different oxyalkylene groups. The copolymer may be a random or block copolymer, for example, a diblock copolymer, or a triblock copolymer. Exemplary oxyalkylene moieties include oxyethylene, oxypropylene, oxytrimethylene, and oxytetramethylene.

An aliphatic polyol may be used as a reactant to form the PEU. Diols are preferred for preparing linear PEUs. Crosslinking can be accomplished by incorporating polyols with more than two reactive groups as a reactant. Exemplary alkylene groups include ethylene, propylene (branched or straight chain), butylene (branched or straight chain), hexylene (branched, straight chain or cyclic) and octylene (branched, straight chain, or cyclic). Exemplary polyols having more than two hydroxyl groups include trimethylolpropane, glycerol, pentaerythritol, 1,2,4-butanetriol, and 2,3,4-pentanetriol. When crosslinking is desired, the tri (or higher)-reactive reactant is used in a minor proportion, e.g., less than 10 equivalent percent of the reactive groups are provided by the cross-linking reactant, and in various embodiments less than 8 or 6 or 4 equivalent percent of the reactive groups are provided by the cross-linking reactant.

An aliphatic polyamine may be used as a reactant to form the PEU. Diamines are preferred for preparing linear PEUs. Crosslinking can be accomplished by incorporating polyamines with more than two reactive groups as a reactant. Exemplary alkylene groups include ethylene, propylene (branched or straight chain), butylene (branched or straight chain), hexylene (branched, straight chain or cyclic) and octylene (branched, straight chain, or cyclic). Exemplary polyamines having more than two amine groups include polypropylenimine tetramine (also known as Dab-Am-4) and triethylenetetramine. The Huntsman Company sells many suitable polyamines having more than two amine groups, for example polyethertriamine (Huntsman product XTJ-566), JEFFAMINE® ST-404 polyetheramine (Huntsman product (XTJ-586), and JEFFAMINE® T-403 polyetheramine. When crosslinking is desired, the tri (or higher)-reactive reactant is used in a minor proportion, e.g., less than 10 equivalent percent of the reactive groups are provided by the cross-linking reactant, and in various embodiments less than 8 or 6 or 4 equivalent percent of the reactive groups are provided by the cross-linking reactant.

An aromatic diol may be used as a reactant to form the PEU. Examples include catechol, resorcinol, hydroquinone and the reactions products thereof, for example, the reaction product of reaction products of resorcinol and ethylene carbonate. Other aromatic diol include bisphenol A and 4,4′-dihydroxybiphenyl.

An aromatic diamine may be used as a reactant to form the PEU. Examples include 1,2-diaminobenzene, 1,3-diaminobenzene, 1,4-diaminobenzene, toluene diamine (e.g., 1,2-diamino-3-methylbenzene, 1,2-diamino-4-methylbenzene, 1,3-diamino-2-methylbenzene, 1,3-diaminoe-4-methylbenzene, 1,4-diamino-2-methylbenzene, 1,4-diamino-3-methylbenzene), alkyl-substituted toluenediamine (e.g., 3,5-diethyltoluene-2,4-diamine and 3,5-diethyltoluene-2,6-diamine), and p-xylyenediamine.

A carbonate may be used as a reactant to form the PEU. Examples include trimethylene carbonate, poly(hexamethylene carbonate)diol, poly(ethylene-carbonate)diol, poly(propylene-carbonate)diol, and poly(butylene-carbonate)diol. When hydroxyl groups are located at either end of a polycarbonate, the material will be referred to herein as a polycarbonate diol.

Glycolide or substituted glycolide may be used as a reactant to form the PEU. The inclusion of glycolide or substituted glycolide among the reactants can achieve formation of ester groups in the PEU. Exemplary substituted glycolides include methyl glycolide (also known as lactide), ethyl glycolide, hexyl glycolide, and isobutyl glycolide.

A blend of polyol or a blend of polyamine may be two or more of aliphatic polyol (or aliphatic polyamine), aromatic polyol (or aromatic polyamine), and polyether polyol (or polyether polyamine).

A polysiloxane may be used as a reactant to form the PEU. Examples include poly(dimethylsiloxane), bis(hydroxylalkyl) terminated and poly(dimethylsiloxane), bis(aminoalkyl) terminated. Additional examples include hydroxyhexyl terminated, hydroxypentyl terminated, and hydroxybutyl terminated polydimethylsiloxane. Polysiloxanes include alkylene chains located between the siloxane group and the terminal hydroxyl (or substituted hydroxyl) group, where an alkylene chain may contain 2 to about 20 methylene groups, for example, 2 to 10 methylene groups.

### Specific PEU Embodiments

Depending on the selection of reactants, the PEU will have various linkage groups and segments.

In one embodiment, the PEU is a polyether urethane (PEUT), i.e., a polymer that contains only urethane as the linking group, and additionally contains polyether segments. The PEUT may be prepared by reaction of a diisocyanate and a polyether diol. Alternatively, the PEUT may be prepared from a polyetherdiisocyanate and an aliphatic diol, e.g., ethylene glycol. The molar ratio of diisocyanate to polyether diol will typically range from 0.95 to 1.05, where the preferred stoichiometric ratio is as close to 1:1 as possible in order to attain the highest molecular weight polymer. Similarly, the molar ratio of polyetherdiisocyanate and aliphatic diol will typically fall within the range of 0.95 to 1.05, and have a preferred stoichiometric ratio of 1:1 in order to attain high molecular weight polymer. The following numbered embodiments provide exemplary PEUT:
1. A polymer composition which is the reaction product of a diisocyanate and a diol.
2. The polymer of embodiment 1 wherein the diol is a polyether diol.
3. The polymer of embodiment 2 wherein the polyether diol comprises at least one type of oxyalkylene sequence selected from the group consisting of oxyethylene, oxypropylene, oxytrimethylene and oxytetramethylene sequences.
4. The polymer of embodiments 1 or 2 wherein the polyetherdiol is a blend of polyetherdiols.
5. The polymer of embodiments 1 or 2 wherein the polyetherdiol is not a blend of polyetherdiols.
6. The polymer of any of embodiments 2-5 wherein the polyetherdiol is a random copolymer of two or more oxyalkylene sequences.
7. The polymer of any of embodiments 2-5 wherein the polyetherdiol is a block copolymer of two or more oxyalkylene sequences.
8. The polymer of any of embodiments 1-7 wherein the diisocyanate is an aliphatic diisocyanate and the reactants do not include an aromatic diisocyanatae.
9. The polymer of any of embodiments 1-7 wherein the diisocyanate is a mixture of aliphatic diisocyanates and the reactants do not include an aromatic diisocyanate.
10. The polymer of any of embodiments 1-7 wherein the diisocyanate is an aromatic diisocyanate and the reactants do not include an aliphatic diisocyanate.
11. The polymer of any of embodiments 1-7 wherein the diisocyanate is a mixture of aromatic diisocyanates and the reactants do not include an aliphatic diisocyanate.
12. The polymer of any of embodiments 1-7 wherein the diisocyanate is a mixture of aromatic diisocyanate and aliphatic diisocyanate.
13. The polymer of any of embodiments 1-12 wherein diisocyanate and diol are the only reactants.
14. The polymer of any of embodiments 2-13 wherein the molar ratio of diisocyanate to polyether diol is in the range of 0.95 to 1.05.

In one embodiment, the PEU is a polyether urea (PEUA), i.e., a polymer that contains only urea as the linking group, and additionally contains polyether segments. The PEUA may be prepared by reaction of a diisocyanate and a polyether diamine. Alternatively, the PEUT may be prepared by reaction of a polyether diisocyanate and an aliphatic diamine, e.g., ethylene diamine. The molar ratio of diisocyanate to polyether diamine will typically range from 0.95 to 1.05, where the preferred stoichiometric ratio is as close to 1:1 as possible in order to attain the highest molecular weight polymer. The following numbered embodiments provide exemplary PEUA:
1. A polymer composition which is the reaction product of a diisocyanate and a diamine.
2. The polymer of embodiment 1 wherein the diamine is a polyether diamine.
3. The polymer of embodiment 2 wherein the polyether diamine comprises at least one type of oxyalkylene sequence selected from the group consisting of oxyethylene, oxypropylene, oxytrimethylene and oxytetramethylene sequences.
4. The polymer of embodiments 1 or 2 wherein the polyetherdiamine is a blend of polyetherdiamines.
5. The polymer of embodiments 1 or 2 wherein the polyether diamine is not a blend of polyetherdiamines.
6. The polymer of any of embodiments 2-5 wherein the polyetherdiamine is a random copolymer of two or more oxyalkylene sequences.
7. The polymer of any of embodiments 2-5 wherein the polyetherdiamine is a block copolymer of two or more oxyalkylene sequences.
8. The polymer of any of embodiments 1-7 wherein the diisocyanate is an aliphatic diisocyanate and the reactants do not include an aromatic diisocyanatae.
9. The polymer of any of embodiments 1-7 wherein the diisocyanate is a mixture of aliphatic diisocyanates and the reactants do not include an aromatic diisocyanate.
10. The polymer of any of embodiments 1-7 wherein the diisocyanate is an aromatic diisocyanate and the reactants do not include an aliphatic diisocyanate.
11. The polymer of any of embodiments 1-7 wherein the diisocyanate is a mixture of aromatic diisocyanates and the reactants do not include an aliphatic diisocyanate.
12. The polymer of any of embodiments 1-7 wherein the diisocyanate is a mixture of aromatic diisocyanate and aliphatic diisocyanate.
13. The polymer of any of embodiments 1-12 wherein diisocyanate and diamine are the only reactants.
14. The polymer of any of embodiments 2-13 wherein the molar ratio of diisocyanate to polyether diamine is in the range of 0.95 to 1.05.

In one embodiment, the PEU is a polyether urea urethane (PEUU), i.e., a polymer that contains both urea and urethane as the only linking groups, and additionally contains polyether segments. The PEUU may be prepared by forming a pre-polymer and then reacting the pre-polymer with either diamine or diol or a mixture thereof. For example, a pre-polymer may be prepared by reacting diisocyanate with polyetherdiol to form a polyether urethane (urethane linkages and polyether segments), and then this pre-polymer is reacted with diamine, e.g., aliphatic diamine, to additionally provide urea linkages and aliphatic segments. Alternatively, a pre-polymer may be prepared by reacting diisocyanate with polyether diamine to form urea linkages and polyether segments with flanking isocyanate end groups, and then this pre-polymer is reacted with diol, e.g., aliphatic diol, to provide urethane linkages and aliphatic segments. A mixture of diol and diamine can also be used, although it should be kept in mind that in most instances the diamine will react more quickly than the diol. The preferred molar ratio of diisocyanate to polyether diol should be about 3:2 for forming the pre-polymer, and the preferred molar ratio of diisocyanate to diamine or diol in the second reaction step is 3:1. Other possible ratios are 2:1 (2:1), 4:3 (4:1), 5:4 (5:1), 6:5 (6:1), 7:6 (7:1), 8:7 (8:1), 9:8 (9:1), and 10:9 (10:1), where the first ratio listed is for diisocyanate to polyether diol in preparation of pre-polymer, and the second ratio in parentheses is the corresponding molar ratio of diisocyanate to diamine or diol for the second reaction step. The following numbered embodiments provide exemplary PEUU:
1. A polymer composition which is the reaction product of a pre-polymer and a diamine, where the pre-polymer is the reaction product of a diisocyanate and a polyetherdiol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences.
7. The polymer of any one of embodiments 1-6 wherein the diamine is an aliphatic diamine.
8. The polymer of any one of embodiments 1-6 wherein the diamine is a polyether diamine.
9. The polymer of any one of embodiments 1-6 wherein the diamine is a blend of diamines.
10. The polymer of any one of embodiments 1-6 wherein the diamine is a blend of aliphatic diamine and polyether diamine.
11. The polymer of any one of embodiments 1-6 wherein the diamine is a blend of aromatic diamine and polyether diamine.
12. The polymer of any one of embodiments 1-6 wherein the diamine is a blend of aromatic diamine and aliphatic diamine.
13. A polymer composition which is the reaction product of a diisocyanate and a polyetherdiamine to form a pre-polymer, and the reaction product of the pre-polymer and a diol to form a polyether urea urethane.
14. The polymer of embodiment 13 wherein the polyetherdiamine comprises at least one type of oxyalkylene sequence selected from the group consisting of oxyethylene, oxypropylene, oxytrimethylene and oxytetramethylene sequences.
15. The polymer of embodiments 13 or 14 wherein the polyetherdiamine is a blend of polyetherdiamines.
16. The polymer of embodiments 13 or 14 wherein the polyetherdiamine is not a blend of polyetherdiamines.
17. The polymer of any one of embodiments 13-16 wherein the polyetherdiamine is a random copolymer of two or more oxyalkylene sequences.
18. The polymer of any one of embodiments 13-16 wherein the polyetherdiamine is a block copolymer of two or more oxyalkylene sequences.
19. The polymer of any one of embodiments 13-18 wherein the diol is an aliphatic diol.
20. The polymer of any one of embodiments 13-18 wherein the diol is an aromatic diol.
21. The polymer of any one of embodiments 13-18 wherein the diol is a polyether diol.
22. The polymer of any one of embodiments 13-18 wherein the diol is a blend of diols.
23. The polymer of any one of embodiments 13-18 wherein the diol is a blend of aliphatic diol and polyetherdiol.
24. The polymer of any one of embodiments 13-18 wherein the diol is a blend of aromatic diol and polyether diol.
25. The polymer of any one of embodiments 13-18 wherein the diol is a blend of aromatic diol and aliphatic diol.
26. The polymer of any one of embodiments 1-25 wherein the diisocyanate is an aliphatic diisocyanate and the reactants do not include an aromatic diisocyanatae to form a polyether urea urethane.
27. The polymer of any one of embodiments 1-25 wherein the diisocyanate is a mixture of aliphatic diisocyanates and the reactants do not include an aromatic diisocyanate to form a polyether urea urethane.
28. The polymer of any one of embodiments 1-25 wherein the diisocyanate is an aromatic diisocyanate and the reactants do not include an aliphatic diisocyanate to form a polyether urea urethane.
29. The polymer of any one of embodiments 1-25 wherein the diisocyanate is a mixture of aromatic diisocyanates and the reactants do not include an aliphatic diisocyanate to form a polyether urea urethane.
30. The polymer of any one of embodiments 1-25 wherein the diisocyanate is a mixture of aromatic diisocyanate and aliphatic diisocyanate to form a polyether urea urethane.

In one embodiment the PEUU is the reaction product of reactants comprising or consisting of a diisocyanate, a diamine and a polyetherdiol, where the diisocyanate is used to form a pre-polymer by reaction with either the diamine or the diol, and then the pre-polymer is used to form the polymer by reaction with the reactant not used to form the pre-polymer, i.e., if the pre-polymer was formed by reaction of diisocyanate and diamine, then the polymer is formed by reaction of pre-polymer and diol, while if the pre-polymer was formed by reaction of diisocyanate and diol, then the polymer is formed by reaction of the pre-polymer and diamine. The term “a” refers to one or more, e.g., a single structure and a blend of different structures. The following two aspects provides examples of this embodiment of the PEUU of the present disclosure.

Accordingly, in one aspect the present disclosure provides a polymer composition which is the reaction product of a pre-polymer and a diamine, where the pre-polymer is the reaction product of a diisocyanate and a polyetherdiol. Optionally, any one or more of the following may be used to further describe this polymer composition and its preparation: the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyetherdiol is a block copolymer of two or more oxyalkylene sequences where this block copolymer may be used as the sole polyetherdiol reactant or it may be used in combination with a different polyetherdiol reactant (e.g., a homopolymeric polyetherdiol formed from oxyalkylene sequences selected from the group consisting of oxyethylene, oxypropylene, oxytrimethylene and oxytetramethylene sequences) to provide a blend which is the polyetherdiol component of the reactants; the diamine is an aliphatic diamine; the diisocyanate is an aliphatic diisocyanate; the polymer is bio-stable; the polymer absorbs at least 50% of its weight in water when immersed in 1% aqueous methyl cellulose at 37° C. for 16 hours; the polymer has a COF of 0.001 to 0.15; the polymer has an intrinsic viscosity of 3-8 dl/g.

A PEUU polymer chain may be described in terms of its structural components rather than in terms of the reactants by which it may be formed. In the afore-described case the polymer chain is a polyurea, having a plurality of urea groups separated alternately by aliphatic groups (contributed by the aliphatic diamine) and polymeric blocks (contributed by the pre-polymer). In other words, the structure may be described by repeating -[urea-aliphatic-urea-polymer block]- units. The polymer block is a polyurethane, having a plurality of urethane (also known as carbamate) groups separated alternatively by aliphatic groups (contributed by the diisocyanate) and polyether groups. In other words, the structure of the polymer block may be described by repeating -[urethane-aliphatic-urethane-polyether]- units. The polyether segments may optionally be selected from oxyethylene, oxypropylene, oxytrimethylene and oxytetramethylene, and in one embodiment the polymer chain contains more than one of these polyether segments, for example, the polymer contains oxyethylene, oxypropylene and oxytetramethylene groups, where optionally the oxyethylene and oxypropylene are arranged in a block copolymer arrangement (e.g., oxyethylene block-oxypropylene block-oxyethylene block). The polymer block may also be referred to as a polyether polyurethane, and the polymer itself may be referred to as a poly ether urethane urea. In one embodiment, the polymer is bio-stable, absorbs at least 50% of its weight in water when immersed in 1% aqueous methyl cellulose at 37° C. for 16 hours, has a COF of 0.001 to 0.15, and has an intrinsic viscosity of 3-8 dl/g.

In a related aspect, the present disclosure provides a PEUU which is the reaction product of a pre-polymer and a polyetherdiol, where the pre-polymer is the reaction product of a diisocyanate and a diamine. Optionally, any one or more of the following may be used to further describe this polymer composition and its preparation: the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyetherdiol is a block copolymer of two or more oxyalkylene sequences where this block copolymer may be used as the sole polyetherdiol reactant or it may be used in combination with a different polyetherdiol reactant (e.g., a homopolymeric polyetherdiol formed from oxyalkylene sequences selected from the group consisting of oxyethylene, oxypropylene, oxytrimethylene and oxytetramethylene sequences) to provide a blend which is the polyetherdiol component of the reactants; the diamine is an aliphatic diamine; the diisocyanate is an aliphatic diisocyanate; the polymer is bio-stable; the polymer absorbs at least 50% of its weight in water when immersed in 1% aqueous methyl cellulose at 37° C. for 16 hours; the polymer has a COF of 0.001 to 0.15; the polymer has an intrinsic viscosity of 3-8 dl/g.

As mentioned previously, a PEUU polymer chain may be described in terms of its structural components rather than in terms of the reactants by which it may be formed. In the afore-described case the PEUU chain is a polyurethane, since it has a plurality of urethane groups separated alternately by polyether groups (contributed by the polyether diol) and polymeric blocks (contributed by the pre-polymer). In other words, the structure may be described by repeating -[urethane-polyether-urethane-polymer block]- units. The polymer block is a polyurea, having a plurality of urea groups separated alternatively by first aliphatic groups (contributed by the diisocyanate) and second aliphatic groups (contributed by the diamine). In other words, the structure of the polymer block may be described by repeating -[urea-first aliphatic-urea-second aliphatic]- units. The polyether segments may optionally be selected from oxyethylene, oxypropylene, oxytrimethylene and oxytetramethylene, and in one embodiment the polymer chain contains more than one of these polyether segments, for example, the polymer contains oxyethylene, oxypropylene and oxytetramethylene groups, where optionally the oxyethylene and oxypropylene are arranged in a block copolymer arrangement (e.g., oxyethylene block-oxypropylene block-oxyethylene block). The polymer block may also be referred to as a polyurea, and the polymer itself may be referred to as a poly ether urethane urea. In one embodiment, the polymer is bio-stable, absorbs at least 50% of its weight in water when immersed in 1% aqueous methyl cellulose at 37° C. for 16 hours, has a COF of 0.001 to 0.15, and has an intrinsic viscosity of 3-8 dl/g.

In one embodiment, the PEU is a polyether carbonate urethane (PECUT), i.e., a polymer that contains urethane linkages, and between the urethane linkages are located a plurality of oxyalkylene groups and a plurality of carbonate groups. In one embodiment, there are two poly(carbonate) groups located between adjacent urethane linkages, where a polyether segment is located between two adjacent poly(carbonate) groups. In one aspect, the weight percent of the combined polyether and polycarbonate segments is 50-99% polycarbonate, or 55-90% polycarbonate, or 60-85% polycarbonate, or 65-75% polycarbonate. The PECUT may be prepared by reacting together a polyether polycarbonate diol, i.e., a diol having a plurality of internal oxyalkyene groups and a plurality of internal carbonate groups, with a diisocyanate. Alternatively, the PECUT may be prepared by reacting together a polyether diol and a polycarbonate diol with a diisocyanate. In either case, the resulting product may be subjected to chain extension with a diol to introduce additional urethane groups. The following numbered embodiments provide exemplary PECUT:
1. A polymer composition which is the reaction product of a diisocyanate and either (a) a mixture comprising polyether diol and polycarbonate diol or (b) a polyether polycarbonate diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences.
7. The polymer of any one of embodiments 1-6 wherein the polycarbonate diol is poly(hexamethylene carbonate)diol.
8. The polymer of any one of embodiments 1-6 wherein the polycarbonate diol is poly(ethylene-carbonate)diol.
9. The polymer of any one of embodiments 1-6 wherein the polycarbonate diol is the reaction product of trimethylene carbonate and a diol.
10. The polymer of any one of embodiments 1-9 wherein the diisocyanate is an aliphatic diisocyanate and the reactants do not include an aromatic diisocyanate.
11. The polymer of any one of embodiments 1-9 wherein the diisocyanate is a mixture of aliphatic diisocyanates and the reactants do not include an aromatic diisocyanate.
12. The polymer of any one of embodiments 1-9 wherein the diisocyanate is an aromatic diisocyanate and the reactants do not include an aliphatic diisocyanate.
13. The polymer of any one of embodiments 1-9 wherein the diisocyanate is a mixture of aromatic diisocyanates and the reactants do not include an aliphatic diisocyanate.
14. The polymer of any one of embodiments 1-9 wherein the diisocyanate is a mixture of aromatic diisocyanate and aliphatic diisocyanate.
15. The polymer of any one of embodiments 1-14 which is further chain extended by reaction with a diol.

In one embodiment, the PEU is a polyether carbonate urethane urea (PECUU), i.e., a polymer that contains urethane linkages as well as urea linkages, and between the urethane linkages are located a plurality of oxyalkylene groups and a plurality of carbonate groups. In one embodiment, there are two poly(carbonate) groups located between adjacent urethane linkages, where a polyether segment is located between two adjacent poly(carbonate) groups. In one aspect, the weight percent of the combined polyether and polycarbonate segments is 50-99% polycarbonate, or 55-90% polycarbonate, or 60-85% polycarbonate, or 65-75% polycarbonate. The PECUU may be prepared by reacting together a polyether polycarbonate diol, i.e., a diol having a plurality of internal oxyalkyene groups and a plurality of internal carbonate groups, with a diisocyanate. Alternatively, the PECUT may be prepared by reacting together a polyether diol and a polycarbonate diol with a diisocyanate. In either case, the resulting product is subjected to chain extension with a diamine to introduce urea groups. The following numbered embodiments provide exemplary PECUU:
1. A polymer composition which is the reaction product of a diamine and a pre-polymer, where the pre-polymer is the reaction product of a diisocyanate and either (a) a mixture comprising polyether diol and polycarbonate diol or (b) a polyether polycarbonate diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences.
7. The polymer of any one of embodiments 1-6 wherein the polycarbonate diol is poly(hexamethylene carbonate)diol.
8. The polymer of any one of embodiments 1-6 wherein the polycarbonate diol is poly(ethylene-carbonate)diol.
9. The polymer of any one of embodiments 1-6 wherein the polycarbonate diol is the reaction product of trimethylene carbonate and a diol.
10. The polymer of any one of embodiments 1-9 wherein the diisocyanate is an aliphatic diisocyanate and the reactants do not include an aromatic diisocyanate.
11. The polymer of any one of embodiments 1-9 wherein the diisocyanate is a mixture of aliphatic diisocyanates and the reactants do not include an aromatic diisocyanate.
12. The polymer of any one of embodiments 1-9 wherein the diisocyanate is an aromatic diisocyanate and the reactants do not include an aliphatic diisocyanate.
13. The polymer of any one of embodiments 1-9 wherein the diisocyanate is a mixture of aromatic diisocyanates and the reactants do not include an aliphatic diisocyanate.
14. The polymer of any one of embodiments 1-9 wherein the diisocyanate is a mixture of aromatic diisocyanate and aliphatic diisocyanate.
15. The polymer of any one of embodiments 1-14 wherein the diamine is an aliphatic diamine.
16. The polymer of any one of embodiments 1-14 wherein the diamine is a polyether diamine.
17. The polymer of any one of embodiments 1-14 wherein the diamine is a blend of diamines.
18. The polymer of any one of embodiments 1-14 wherein the diamine is a blend of aliphatic diamine and polyether diamine.

In one embodiment, the PEU is a polyether ester urethane (PEEUT), i.e., a polymer that contains urethane linkages, and between the urethane linkages are located polyether and polyester groups. In one embodiment, a single block of polyether and a single block of polyester are located between urethane linkages. The PEEUT may be prepared by forming a pre-polymer of polyether and polyester having flanking hydroxyl groups. The pre-polymer is then reacted with diisocyanate to form urethane linkages on either side of the polyether polyester diblock. The following numbered embodiments provide exemplary PEEUT:
1. A polymer composition which is the reaction product of a diisocyanate and either (a) a mixture comprising polyether diol and polyester diol or (b) a polyether polyester diol.
2. The polymer of embodiment 1 wherein the polyether diol comprises at least one type of oxyalkylene sequence selected from the group consisting of oxyethylene, oxypropylene, oxytrimethylene and oxytetramethylene sequences.
3. The polymer of embodiments 1 or 2 wherein the polyether diol is a blend of polyetherdiols.
4. The polymer of embodiments 1 or 2 wherein the polyether diol is not a blend of polyetherdiols.
5. The polymer of any one of embodiments 1-4 wherein the polyether diol is a random copolymer of two or more oxyalkylene sequences.
6. The polymer of any one of embodiments 1-4 wherein the polyether diol is a block copolymer of two or more oxyalkylene sequences.
7. The polymer of any one of embodiments 1-6 wherein the diisocyanate is an aliphatic diisocyanate and the reactants do not include an aromatic diisocyanate.
8. The polymer of any one of embodiments 1-6 wherein the diisocyanate is a mixture of aliphatic diisocyanates and the reactants do not include an aromatic diisocyanate.
9. The polymer of any one of embodiments 1-6 wherein the diisocyanate is an aromatic diisocyanate and the reactants do not include an aliphatic diisocyanate.
10. The polymer of any one of embodiments 1-6 wherein the diisocyanate is a mixture of aromatic diisocyanates and the reactants do not include an aliphatic diisocyanate.
11. The polymer of any one of embodiments 1-6 wherein the diisocyanate is a mixture of aromatic diisocyanate and aliphatic diisocyanate.

In one embodiment, the PEU is a polyether ester urethane urea (PEEUU), i.e., a polymer than contains both urethane and urea linkages, and between those urethane and urea linkages are located polyether and polyester groups. The following numbered embodiments provide exemplary PEEUU:
1. A polymer composition which is the reaction product of a diamine and a pre-polymer, where the pre-polymer is the reaction product of a diisocyanate and either (a) a mixture comprising polyether diol and polyester diol or (b) a polyether polyester diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences.
7. The polymer of any one of embodiments 1-6 wherein the diisocyanate is an aliphatic diisocyanate and the reactants do not include an aromatic diisocyanate.
8. The polymer of any one of embodiments 1-6 wherein the diisocyanate is a mixture of aliphatic diisocyanates and the reactants do not include an aromatic diisocyanate.
9. The polymer of any one of embodiments 1-6 wherein the diisocyanate is an aromatic diisocyanate and the reactants do not include an aliphatic diisocyanate.
10. The polymer of any one of embodiments 1-6 wherein the diisocyanate is a mixture of aromatic diisocyanates and the reactants do not include an aliphatic diisocyanate.
11. The polymer of any one of embodiments 1-6 wherein the diisocyanate is a mixture of aromatic diisocyanate and aliphatic diisocyanate.
12. The polymer of any one of embodiments 1-11 wherein the diamine is an aliphatic diamine.
13. The polymer of any one of embodiments 1-11 wherein the diamine is a polyether diamine.
14. The polymer of any one of embodiments 1-11 wherein the diamine is a blend of diamines.
15. The polymer of any one of embodiments 1-11 wherein the diamine is a blend of aliphatic diamine and polyether diamine.

In one embodiment, the PEU is a polyether siloxane urethane (PESUT), i.e., a polymer that contains urethane linkages, and between the urethane linkages are located a plurality of oxyalkylene groups and a plurality of siloxane groups. In one embodiment, there are two poly(siloxane) groups located between adjacent urethane linkages, where a polyether segment is located between two adjacent poly(siloxane) groups. In one aspect, the weight percent of the combined polyether and polysiloxane segments is 5-50% polysiloxane, or 10-40% polysiloxane, or 10-30% polysiloxane, or 50-99% polysiloxane, or 55-90% polysiloxane, or 60-85% polysiloxane or 65-75% polysiloxane. The PESUT may be prepared by reacting together a polyether diol and a polysiloxane diol with a diisocyanate. In every case, the resulting product may be subjected to chain extension with a diol to introduce additional urethane groups. The following numbered embodiments provide exemplary PESUT:
1. A polymer composition which is the reaction product of a diisocyanate and either (a) a mixture comprising polyether diol and polysiloxane diol or (b) a polyether polysiloxane diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences.
7. The polymer of any one of embodiments 1-6 wherein the polysiloxane diol is poly(dimethylsiloxane), bis(hydroxyalkyl) terminated.
8. The polymer of any one of embodiments 1-6 wherein the polysiloxane diol is hydroxyhexyl terminated polydimethylsiloxane.
9. The polymer of any one of embodiments 1-6 wherein the polysiloxane diol is hydroxypentyl terminated polydimethylsiloxane.
10. The polymer of any one of embodiments 1-9 wherein the diisocyanate is an aliphatic diisocyanate and the reactants do not include an aromatic diisocyanatae.
11. The polymer of any one of embodiments 1-9 wherein the diisocyanate is a mixture of aliphatic diisocyanates and the reactants do not include an aromatic diisocyanate.
12. The polymer of any one of embodiments 1-9 wherein the diisocyanate is an aromatic diisocyanate and the reactants do not include an aliphatic diisocyanate.
13. The polymer of any one of embodiments 1-9 wherein the diisocyanate is a mixture of aromatic diisocyanates and the reactants do not include an aliphatic diisocyanate.
14. The polymer of any one of embodiments 1-9 wherein the diisocyanate is a mixture of aromatic diisocyanate and aliphatic diisocyanate.
15. The polymer of any one of embodiments 1-14 which is further chain extended by reaction with a diol.

In one embodiment, the PEU is a polyether siloxane urethane urea (PESUU), i.e., a polymer that contains urethane linkages as well as urea linkages, and between the urethane linkages are located a plurality of oxyalkylene groups and a plurality of siloxane groups. In one embodiment, there are two poly(siloxane) groups located between adjacent urethane linkages, where a polyether segment is located between two adjacent poly(siloxane) groups. In one aspect, the weight percent of the combined polyether and polysiloxane segments is 5-50% polysiloxane, or 10-40% polysiloxane, or 10-30% polysiloxane, or 50-99% polysiloxane, or 55-90% polysiloxane, or 60-85% polysiloxane, or 65-75% polysiloxane. The PESUU may be prepared by reacting together a polyether diol and a polysiloxane diol with a diisocyanate. Alternatively, the PESUU may be prepared by reacting together a polyether diol and a polysiloxane diamine with a diisocyanate. In every case, the resulting product may be subjected to chain extension with a diamine to introduce urea groups. The following numbered embodiments provide exemplary PESUU:
1. A polymer composition which is the reaction product of a diamine and a pre-polymer, where the pre-polymer is the reaction product of a diisocyanate and either (a) a mixture comprising polyether diol and polysiloxane diol or (b) a polyether polysiloxane diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences.
7. The polymer of any one of embodiments 1-6 wherein the polysiloxane diol is poly(dimethylsiloxane), bis(hydroxyalkyl) terminated.
8. The polymer of any one of embodiments 1-6 wherein the polysiloxane diol is hydroxyhexyl terminated polydimethylsiloxane.
9. The polymer of any one of embodiments 1-6 wherein the polysiloxane diol is hydroxypentyl terminated polydimethylsiloxane.
10. The polymer of any one of embodiments 1-9 wherein the diisocyanate is an aliphatic diisocyanate and the reactants do not include an aromatic diisocyanate.
11. The polymer of any one of embodiments 1-9 wherein the diisocyanate is a mixture of aliphatic diisocyanates and the reactants do not include an aromatic diisocyanate.
12. The polymer of any one of embodiments 1-9 wherein the diisocyanate is an aromatic diisocyanate and the reactants do not include an aliphatic diisocyanate.
13. The polymer of any one of embodiments 1-9 wherein the diisocyanate is a mixture of aromatic diisocyanates and the reactants do not include an aliphatic diisocyanate.
14. The polymer of any one of embodiments 1-9 wherein the diisocyanate is a mixture of aromatic diisocyanate and aliphatic diisocyanate.
15. The polymer of any one of embodiments 1-14 wherein the diamine is an aliphatic diamine.
16. The polymer of any one of embodiments 1-14 wherein the diamine is a polyether diamine.
17. The polymer of any one of embodiments 1-14 wherein the diamine is a blend of diamines.
18. The polymer of any one of embodiments 1-14 wherein the diamine is a blend of aliphatic diamine and polyether diamine.

The following descriptions 1-14 provides additional embodiments of PEU as well as describing component parts of embodiments of PEU, where these additional embodiments may be used to further describe or characterize the PEUs identified herein, e.g. a polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), and polyether siloxane urethane urea (PESUU).
1. A hydroswellable, segmented, aliphatic polyurethane PEU composition, comprising at least a first and a second different polyoxyalkelene chain segment covalently linked by a second group of chain segments including aliphatic urethane segments, wherein said composition swells at least 5% when immersed in water. Such a PEU may be prepared by reacting a polyoxyalkylene (with hydroxyl end groups) with diisocyanate to create a high molecular weight polymer.
2. A hydroswellable, segmented, aliphatic polyether-urea PEU composition, comprising at least a first and a second different polyoxyalkylene chain segment covalently linked by a second group of chain segments including aliphatic urea segments, wherein said composition swells at least 5% when immersed in water. Such a PEU could be prepared by reacting a polyetheramine with diisocyanate to create urea linkages in the absence of urethane linkages (the latter of which are the byproduct of reacting hydroxyl groups with isocyanate groups).
3. A hydroswellable, segmented, aliphatic polyetherurethane-urea PEU composition, comprising at least a first and a second different polyoxyalkylene chain segment covalently linked to form a multiblock copolymer, which is covalently linked by a second group of chain segments including both aliphatic urethane and urea segments, wherein said composition swells at least 5% when immersed in water.
4. A hydroswellable, segmented, aliphatic polyurethane PEU composition, comprising at least one polyoxyalkylene chain segment and one polycarbonate chain segment covalently linked by a second group of chain segments including both aliphatic urethane and urea segments, wherein said composition swells at least 5% when immersed in water.
5. A hydroswellable, segmented, aliphatic polyurethane-urea PEU composition, comprising at least one polyoxyalkelene chain segment and one polycarbonate chain segment covalently linked by a second group of aliphatic urethane segments, wherein said composition swells at least 5% when immersed in water.
6. A hydroswellable, segmented, aliphatic polyurethane PEU composition, comprising at least one polyoxyalkylene chain segment and one polysiloxane chain segment covalently linked by a second group of chain segments including both aliphatic urethane and urea segments, wherein said composition swells at least 5% when immersed in water.
7. A hydroswellable, segmented, aliphatic polyurethane-urea PEU composition, comprising at least one polyoxyalkelene chain segment and one polysiloxane chain segment covalently linked by a second group of aliphatic urethane segments, wherein said composition swells at least 5% when immersed in water.
8. A PEU wherein a polyoxyalkylene chain segment comprises segments derived from at least one polyoxyalkylene selected from the group consisting of poly(ethylene glycol), poly(propylene glycol), poly(ethylene glycol)-block-poly(propylene glycol), poly(propylene glycol)-block-poly(ethylene glycol)-block-poly(propylene glycol), and poly(ethylene glycol-ran-propylene glycol), and optionally including poly(ethylene glycol)-block-poly(propylene glycol)-block-poly(ethylene glycol).
9. A PEU wherein polyoxyalkylene chain segment comprises segments derived from at least one polyoxyalkylene selected from the group consisting of poly(tetramethylene glycol-block-ethylene glycol), poly(tetramethylene glycol-block-propylene glycol), poly(ethylene glycol-block-tetramethylene glycol-block-ethylene glycol), poly(trimethylene glycol), poly(pentamethylene glycol), and poly(hexamethylene glycol), and optionally including poly(tetramethylene glycol).
10. A PEU wherein urethane segments are derived from at least one diisocyanate selected from the group consisting of isophorone diisocyanate, 4,4′-methylenediphenyl diisocyanate, m-phenylene diisocyanate, p-phenylene diisocyanate, 2,6-TDI, 2,4-TDI, 1,5-Naphthalene diisocyanate, 4.4-MDI, 2.4-MDI, 2,2,-MDI, MDI, Tolidine diisocyanate, dianisidine diisocyanate, p-xylene diisocyanate, m-xylene diisocyanate, m-TMXDI (1,3-Bis(1-isocyanato-1-methylethyl)benzene), p-TMXDI (1,4-Bis(1-isocyanato-1-methylethyl)benzene), 1,5-diisocyanato-2-methylpentane, lysine diisocyanate methyl ester, 2,2,4-trimethylhexane 1,6-diisocyanate, 2,4,4-trimethylhexane 1,6-diisocyanate, 2,5(6)-Bis(isocyanatomethyl)bicycle[2.2.1]heptane, 1,3,3-trimethyl-1-(isocyanatomethyl)-5-isocyanatocyclohexane, 1,3-diisocyanato-2,4-dimethyloctane, octahydro-4,7-methano-1H-indenedimethyl diisocyanate, 1,1′-methylenebis(4-isocyanatocyclohexane), poly(ethylene oxide)diisocyanate, and poly(propylene oxide)diisocyanate, and wherein the resulting polyoxyalkylene urethane molecules have isocyanate terminal groups that are chain-extended with an alkylene diamine selected from the group consisting of ethylene-, trimethylene-, tetramethylene-, hexamethylene-, and octamethylene-diamine, thereby forming polyetherurethane-urea segmented chains.
11. A PEU which comprises urethane segments derived from at least one diisocyanate selected from the group consisting of hexamethylene diisocyanate, octamethylene diisocyanate, decamethylene diisocyanate, dodecamethylene diisocyanate, 1,4-cyclohexane diisocyanate, and cyclohexane bis(methylene isocyanate), and wherein the resulting polyoxyalkylene urethane molecules have isocyanate terminal groups that are chain-extended with a polyetheramine selected from the group consisting of poly(ethylene glycol)diamine, poly(propylene glycol)diamine, poly(ethylene-co-propylene glycol)diamines, poly(trimethylene glycol)diamine, poly(tetramethylene glycol)diamines, poly(pentamethylene glycol)diamines, and poly(hexamethylene glycol)diamine, thereby forming polyetherurethane-urea segmented chains.
12. A PEU which comprises urethane segments derived from at least one diisocyanate selected from the group consisting of isophorone diisocyanate, 4,4′-methylenediphenyl diisocyanate, m-phenylene diisocyanate, p-phenylene diisocyanate, 2,6-TDI, 2,4-TDI, 1,5-Naphthalene diisocyanate, 4.4-MDI, 2.4-MDI, 2,2,-MDI, MDI, Tolidine diisocyanate, dianisidine diisocyanate, p-xylene diisocyanate, m-xylene diisocyanate, m-TMXDI (1,3-Bis(1-isocyanato-1-methylethyl)benzene), p-TMXDI (1,4-Bis(1-isocyanato-1-methylethyl)benzene), 1,5-diisocyanato-2-methylpentane, lysine diisocyanate methyl ester, 2,2,4-trimethylhexane 1,6-diisocyanate, 2,4,4-trimethylhexane 1,6-diisocyanate, 2,5(6)-Bis(isocyanatomethyl)bicycle[2.2.1]heptane, 1,3,3-trimethyl-1-(isocyanatomethyl)-5-isocyanatocyclohexane, 1,3-diisocyanato-2,4-dimethyloctane, octahydro-4,7-methano-1H-indenedimethyl diisocyanate, 1,1′-methylenebis(4-isocyanatocyclohexane), poly(ethylene oxide)diisocyanate, and poly(propylene oxide)diisocyanate, and wherein the resulting polyoxyalkylene urethane molecules have isocyanate terminal groups that are chain-extended with a polyetheramine selected from the group consisting of poly(ethylene glycol)diamine, poly(propylene glycol)diamine, poly(ethylene-co-propylene glycol)diamines, poly(trimethylene glycol)diamine, poly(tetramethylene glycol)diamines, poly(pentamethylene glycol)diamines, and poly(hexamethylene glycol)diamine, thereby forming polyetherurethane-urea segmented chains.
13. A PEU which comprises polycarbonate chain segments derived from at least one polycarbonate selected from the group consisting of poly(1,6-hexyl-1,2-ethyl-carbonate)diol, poly(1,6-hexyl-carbonate)diol, poly(1,2-ethyl-carbonate)diol, poly(1,4-butyl-carbonate)diol, poly(1,5-pentyl-carbonate)diol, and poly(trimethylene carbonate)diol.
14. A PEU which comprises polysiloxane chain segments derived from at least one polysiloxane selected from the group consisting of poly(dimethylsiloxane), bis(hydroxylalkyl) terminated and poly(dimethylsiloxane), bis(aminoalkyl) terminated. Additional examples include hydroxyhexyl terminated, hydroxypentyl terminated, and hydroxybutyl terminated polydimethylsiloxane. Polysiloxanes include alkylene chains located between the siloxane group and the terminal hydroxyl (or substituted hydroxyl) group, where an alkylene chain may contain 2 to about 20 methylene groups, for example, 2 to 10 methylene groups.

### PEU Properties

The PEU may be described in terms of its properties in addition to, or instead of, being described in terms of its chemical composition and/or its method of manufacture. One or more of the following properties may be used to characterize any of the PEU or specific PEU embodiments described herein, where in various aspects each property used to characterize a PEU may have a value or range of values as stated below.

In one embodiment, the PEU may be characterized by the extent to which it absorbs water. For instance, the PEU may be hydroswellable, or in other words, when a PEU sample of a specified volume is placed into pure water, the sample will absorb water and swell to a larger volume. In various aspects, the PEU swells to a volume that is at least 10%, or at least 20%, or at least 30%, or at least 40%, or at least 50%, or at least 60%, or at least 70%, or at least 80%, greater than its initial volume. In various aspects, suitable ranges are 40-80% swelling, 50-70% swelling, or 55-65% swelling.

Alternatively, or additionally, the extent to which a PEU absorbs water may be measured on a mass basis. A suitable test for measuring water absorption is to prepare film strips that are weighed to determine an initial weight. The strips are submerged in a solution of 1% methyl cellulose (dissolved in deionized water) for 16 hours at 37° C. The film strips are removed and blotted dry, and the final weight is recorded. The final weight is subtracted from the initial weight, and the difference is divided by the initial weight and then multiplied by 100 to determine the percentage of water that is absorbed by the film strips relative to the initial weight. In various aspects, the water uptake is greater than 50%, or greater than 55%, or greater than 60%, or greater than 65%, or greater than 70%, or greater than 75%, or greater than 80%, or greater than 85%, or greater than 90%.

The extent to which a PEU swells may also be measured in terms of a change in thickness of a sample of PEU when that sample is exposed to moisture. A suitable test method to measure increase in thickness due to swelling is to prepare film strips and then determine their initial dimensions, specifically the thickness. The strips are submerged in a solution of 1% methyl cellulose (dissolved in deionized water) for 16 hours at 37° C. The film strips are removed and blotted dry, and the final thickness is measured. The final thickness is subtracted from the initial thickness, and the difference is divided by the initial thickness and then multiplied by 100 to determine the percent increase in thickness of the film strips relative to the initial dimension. In various aspects, the increase in thickness is greater than 5%, or greater than 10%, or greater than 15%.

In one embodiment, the PEU may be characterized in terms of whether, or the extent to which, the PEU absorbs or degrades or is structurally stable in a biological environment. In one embodiment, the PEU is non-absorbable, or in other words, is bio-stable. A bio-stable PEU is particularly useful where implantation of PEU is desired for long-term performance, e.g., as cartilage replacement. As used herein, a PEU is bio-stable if it experiences less than 5% weight loss over a six month period when exposed to biological fluid. When some degree of absorbable performance is desired of the PEU, a polyester segment made from, e.g., glycolide or substituted glycolide, may be included in the PEU.

In one embodiment, the PEU may be characterized by its inherent viscosity. For example, the inherent viscosity of a PEU may be measured according to the procedure described in ASTM D2857-95. In various aspects, the inherent viscosity of the PEU is greater than 2 dl/g, or greater than 2.5 dl/g, or greater than 3 dl/g, or greater than 3.5 dl/g, or greater than 4 dl/g, or greater than 4.5 dl/g, or greater than 5 dl/g. In various aspects, the inherent viscosity may be as high as 10 dl/g, or as high as 9 dl/g, or as high as 8 dl/g, or as high as 7 dl/g. Thus, suitable exemplary ranges are 2-10 dl/g, or 3-8 dl/g, or 4-7 dl/g.

In one embodiment, the PEU may be characterized by its coefficient of friction (COF). COF may be measured according to the procedure described in ASTM D1894. In various aspects, the COF of the PEU is less than 0.2, or less than 0.15, or less than 0.1, or less than 0.05, or less than 0.03. In other aspects, the COF is within the range of 0.001 to 0.20, or within the range of 0.001 to 0.18, or within the range of 0.001 to 0.15, or within the range of 0.005 to 0.10.

In one embodiment, the PEU may be characterized by its burst properties. For example, the burst strength of the PEU may be measured according to a modified version of ASTM D3787-07, Standard Test Method for Bursting Strength of Textiles-Constant-Rate-of-Traverse (CRT) Ball Burst Test, in which the modified version of this method is conducted using a testing apparatus that is an MTS Synergie equipped with a ball burst test fixture. The fixture consists of an upper ball portion and a lower fixture plate for securing the film sample, wherein the upper ball portion is a plunger of diameter 11.4 mm and the lower fixture plate has a circular hold of diameter 20 mm for accepting said plunger. The ball portion of the test fixture is attached to the MTS Synergie and the system is zeroed to account for the mass of the fixture. The top clamp of the fixture plate is removed and the film sample with a thickness of approximately 0.60 mm is placed on the ball burst fixture base. Next, the sample is centered within the threaded holes used to attach the top clamp plate. The top clamp plate is attached over the film, and the sample is secured in the fixture by tightening the four socket head cap screws using an allen wrench ( 3/16″). The test is initiated by manually lowering the upper ball portion of the test fixture to contact the film, providing a 0.1 N preload. The plunger is lowered at a rate of 1 inch per minute onto the film sample until the film fails, at which point the ball portion penetrates the opening in the lower fixture plate to complete the test. Under these test conditions, in various aspects, the PEU may have a minimum extension (measured at peak load during burst testing using a wet sample of PEU) of greater than 30 mm, or greater than 35 mm, or greater than 40 mm, or greater than 45 mm, or greater than 50 mm, or greater than 55 mm, or greater than 60 mm, or greater than 65 mm, or greater than 70 mm. This same test method may be used to measure the peak load of a wet PEU sample, where in various aspects the peak load is greater than 70 N, or greater than 75 N, or greater than 80 N, or greater than 85 N, or greater than 90 N, or greater than 95 N, or greater than 100 N, or greater than 105 N, or greater than 110 N, or greater than 115N, or greater than 120 N, or greater than 125 N, or greater than 130 N.

In one embodiment, the PEU may be analyzed by differential scanning calorimetry (DSC) and/or associated thermal transitions. To make such analysis, samples weighing approximately 5-10 milligrams are loaded into a differential scanning calorimeter and heated at a controlled rate (e.g. 10° C./min, 15° C./min, or 20° C./min) from 0° C. to 230° C. The sample can be quenched by immediate cooling in liquid nitrogen, or can be cooled at a controlled rate (e.g. 10° C./min, 15° C./min, or 20° C./min) to a reduced temperature at or below room temperature. Upon cooling, the sample can be reheated at a controlled rate (10° C./min, 15° C./min, or 20° C./min) to 230° C. in order to obtain thermal data reflecting the absence of a thermal history. This type of method provides data related to both the thermal history of the sample (first run) and data that also reflects the absence of a thermal history (second run). Under these test conditions, in various aspects, the PEU may have an endothermic phase transition (melting event) below 100° C., or below 80° C., or below 70° C., or below 65° C., or below 60° C., or below 55° C. This same test method may be used to measure the heat of melting of a PEU soft segment, where in various aspects the heat of melting is between 1 joules/gram and 50 joules/gram, or between 5 joules/gram and 40 joules/gram, or between 5 joules/gram and 30 joules/gram, or between 5 joules per gram and 25 joules/gram, or between 10 joules per gram and 25 joules/gram.

Some exemplary PEU and their corresponding properties are provided in the Table.

TABLE

| | | | Swelling | | Percent | | Burst Test: |
|---|---|---|---|---|---|---|---|
| | | | (% water | | Poly- | Burst Test: | Max |
| Polymer | Percent | I.V.² | uptake, by | | carbonate | Peak Load⁵ | Extension⁶ |
| Class | Polyether¹ | (dL/g) | mass)³ | COF | (siloxane)⁴ | (N) | (mm) |
| Polyether | 50-99% | >2.0 dl/g | >30% | <.15 | NA | >80 N | >40 mm |
| Urethane | 65-99% | >3.5 dl/g | >40% | <.10 | | >90 N | >50 mm |
| (PEUT) | 75-99% | >5.0 dl/g | >50% | <.05 | | >120 N | >60 mm |
| | 85-99% | | | | | | |
| Polyether Urea | 50-99% | >2.0 dl/g | >30% | <.15 | NA | >80 N | >40 mm |
| (PEUA) | 65-99% | >3.5 dl/g | >40% | <.10 | | >90 N | >50 mm |
| | 75-99% | >5.0 dl/g | >50% | <.05 | | >120 N | >60 mm |
| | 85-99% | | | | | | |
| Polyether | 50-99% | >2.0 dl/g | >30% | <.15 | NA | >80 N | >40 mm |
| Urethane Urea | 65-99% | >3.5 dl/g | >40% | <.10 | | >90 N | >50 mm |
| (PEUU) | 75-99% | >5.0 dl/g | >50% | <.05 | | >120 N | >60 mm |
| | 85-99% | | | | | | |
| Polyether | 50-99% | >2.0 dl/g | >30% | <.15 | 50-99% | >80 N | >40 mm |
| Carbonate | 65-99% | >3.5 dl/g | >40% | <.10 | 55-90% | >90 N | >50 mm |
| Urethane Urea | 75-99% | >5.0 dl/g | >50% | <.05 | 60-85% | >120 N | >60 mm |
| (PECUU) | 85-99% | | | | 65-75% | | |
| Polyether | 50-99% | >2.0 dl/g | >30% | <.15 | 50-99% | >80 N | >40 mm |
| Siloxane | 65-99% | >3.5 dl/g | >40% | <.10 | 55-90% | >90 N | >50 mm |
| Urethane Urea | 75-99% | >5.0 dl/g | >50% | <.05 | 60-85% | >120 N | >60 mm |
| (PESUU) | 85-99% | | | | 65-75% | | |

¹Per repeat unit, by mass
²Inherent viscosity
³% water uptake, by mass
⁴% of total soft segments, by mass
⁵Peak load determined from burst testing; performed on wet test specimen
⁶Maximum extension measured at peak load during burst testing; performed on wet test specimen

As stated above, these properties may be used to further describe or characterize the PEUs identified herein, e.g. a polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), and polyether siloxane urethane urea (PESUU).

### Method of Making PEU Polymers

The PEU polymers may be prepared by reacting a diisocyanate with one or both of a diol and a diamine. The diol may be, for example, a polyether diol, i.e., a polyether segment flanked by two hydroxyl groups, to thereby provide for incorporation of polyether functionality into the PEU. The diol may be a polyether polyester diol, i.e., a polyether segment joined to a polyester segment where the two segments are flanked by two hydroxyl groups, to thereby provide for incorporation of both polyether and polyester functionality into the PEU. The diol may be a polyether carbonate diol, i.e., a polyether segment that is joined to at least two carbonate groups, the polyether carbonate diol having two terminal hydroxyl groups to thereby provide for incorporation of both polyether and polycarbonate functionality into the PEU.

Representative examples of synthesis techniques that may be adapted to prepare PEUs are provided in US 2010/0056646 and US 2009/0233887, both of which are incorporated by reference in their entirety.

The PEU may be sterilized prior to, or preferably after, being packaged for shipment. For example, the PEU may be exposed to radiation such as gamma rays or E-beams for a sufficient period of time to achieve sterilization. Alternatively, or additionally, the PEU may be sterilized by exposing the PEU to chemical sterilization agents, e.g., ethylene oxide.

These methods are applicable to the PEUs identified herein, e.g. a polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), and polyether siloxane urethane urea (PESUU).

### Forming Desired Shapes

The PEU may be formed into a film or sheet, or other suitable shape. Suitable shapes may be achieved preferably by dip-coating of a mold into a liquid solution containing the PEU. Dip-coating can be performed through a method involving multiple dips of the mold into a liquid solution of the PEU, wherein the polymer is dissolved in a fluorinated solvent that is highly volatile, such as trifluorethanol (TFE) or hexafluoroisopropanol (HFIP). The mold is dipped multiple times until the appropriate thickness of the final film is formed on the surface of the mold. Another option for forming a desired shape of PEU is to prepare the PEU within a mold of a desired shape.

These shapes are applicable to the PEUs identified herein, e.g. a polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), polyether ester urethane urea (PEEUU), polyether siloxane urethane (PESUT), and polyether siloxane urethane urea (PESUU).

### Bioactive Agents

The PEU polymers of the present application may be in combination with one or more bioactive agents. The bioactive agents may be incorporated into or onto the PEU polymers by a variety of methods, including for example, by applying the bioactive agent to the polymer (e.g., coating, painting, dipping or spraying the polymer onto one or more surfaces or a portion of a surface of the polymer), and/or by incorporating the bioactive agent, or a composition comprising the bioactive agent within the polymer (e.g., by admixing the bioactive agent within the polymer, or a portion of the polymer during formation of the PEU, or by admixing the bioactive agent with one or more polymers and incorporating these polymers into the PEU polymer). Since the PEU polymers are hydroswellable, bioactive agent may be incorporated into the PEU at the same time that PEU absorbs water. For example, the bioactive agent may be dissolved in a saline/water solution, or may be formed into an aqueous dispersion of liposome or micelle in the case of a hydrophobic bioactive agent. The PEU polymer may then be placed into the bioactive agent solution/dispersion, and the bioactive agent will enter the PEU polymer along with the water. Within certain embodiments the bioactive agent is designed to be released from the PEU polymer over a desired time frame.

Examples of such bioactive agents includes, but are not limited to, fibrosis-inducing agents, antifungal agents, antibacterial agents and antibiotics, anti-inflammatory agents, anti-scarring agents, immunosuppressive agents, immunostimulatory agents, antiseptics, anesthetics, antioxidants, cell/tissue growth promoting factors, anti-neoplastic, anticancer agents and agents that support ECM integration.

Examples of fibrosis-inducing agents include, but are not limited to talcum powder, metallic beryllium and oxides thereof, copper, silk, silica, crystalline silicates, talc, quartz dust, and ethanol; a component of extracellular matrix selected from fibronectin, collagen, fibrin, or fibrinogen; a polymer selected from the group consisting of polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)-methylated collagen; an inflammatory cytokine (e.g., TGF, PDGF, VEGF, bFGF, TNFα, NGF, GM-CSF, IGF-a, IL-1, IL-1-, IL-8, IL-6, and growth hormone); connective tissue growth factor (CTGF); a bone morphogenic protein (BMP) (e.g., BMP-2, BMP-3, BMP-4, BMP-5, BMP-6, or BMP-7); leptin, and bleomycin or an analogue or derivative thereof.

Optionally, the PEU polymer may additionally comprise a proliferative agent that stimulates cellular proliferation. Examples of proliferative agents include: dexamethasone, isotretinoin (13-cis retinoic acid), 17-1-estradiol, estradiol, 1-a-25 dihydroxyvitamin D3, diethylstibesterol, cyclosporine A, L-NAME, all-trans retinoic acid (ATRA), and analogues and derivatives thereof. (see US 2006/0240063, which is incorporated by reference in its entirety).

Examples of antifungal agents include, but are not limited to, polyene antifungals, azole antifungal drugs, and Echinocandins.

Examples of antibacterial agents and antibiotics include, but are not limited to, erythromycin, penicillins, cephalosporins, doxycycline, gentamicin, vancomycin, tobramycin, clindamycin, and mitomycin.

Examples of anti-inflammatory agents include, but are not limited to, non-steroidal anti-inflammatory drugs such as ketorolac, naproxen, diclofenac sodium and fluribiprofen.

Examples of anti-scarring agents include, but are not limited to cell-cycle inhibitors such as a taxane, immunomodulatory agents such as serolimus or biolimus (see, e.g., paras. 64 to 363, as well as all of US 2005/0149158, which is incorporated by reference in its entirety).

Examples of immunosuppressive agents include, but are not limited to, glucocorticoids, alkylating agents, antimetabolites, and drugs acting on immunophilins such as ciclosporin and tacrolimus.

Examples of immunostimulatory agents include, but are not limited to, interleukins, interferon, cytokines, toll-like receptor (TLR) agonists, cytokine receptor agonist, CD40 agonist, Fe receptor agonist, CpG-containing immunostimulatory nucleic acid, complement receptor agonist, or an adjuvant.

Examples of antiseptics include, but are not limited to, chlorhexidine and tibezonium iodide.

Examples of anesthetic include, but are not limited to, lidocaine, mepivacaine, pyrrocaine, bupivacaine, prilocalne, and etidocaine.

Examples of antioxidants include, but are not limited to, antioxidant vitamins, carotenoids, and flavonoids.

Examples of cell growth promoting factors include, but are not limited to, epidermal growth factors, human platelet derived TGF-, endothelial cell growth factors, thymocyte-activating factors, platelet derived growth factors, fibroblast growth factor, fibronectin or laminin.

Examples of antineoplastic/anti-cancer agents include, but are not limited to, paclitaxel, carboplatin, miconazole, leflunamide, and ciprofloxacin.

Examples of agents that support ECM integration include, but are not limited to, gentamicin.

It is recognized that in certain forms of therapy, combinations of agents/drugs in the same PEU polymer can be useful in order to obtain an optimal effect. Thus, for example, an antibacterial and an anti-inflammatory agent may be combined into PEU in order to provide combined effectiveness. Particularly preferred combinations for use within the present invention include a combination of anti-inflammatory and anesthetics, or a combination of anti-inflammatory, anesthetic, and anti-bacterial agents. In some embodiments, one or more bioactive agents (e.g., a fibrosis-inducing drug) are applied to only a specific section or area of the PEU polymer, as opposed to the entire polymer. In other embodiments, two or more drugs are applied to two or more areas of the polymer.

### Method of Applying PEU to a Substrate

Within certain embodiments of the invention, methods are provided for applying PEU to a desired substrate. Representative examples of suitable substrates include, for example, medical devices, as well as biological surfaces (such as the femoral head).

The PEU polymers of the present invention may be applied to a wide variety of medical devices. Particularly preferred medical devices include artificial joints, including for example, hip joints, knee joints, and the temporomandibular joint. Within certain embodiments of the invention, the PEU polymer is formed as a film, sheet, or cap to fit over the surface of bony structures (e.g., femoral head of the femoral joint), particularly in joints where articular cartilage has degenerated. Preferably, the polymer is formed to help protect damaged, injured, surgically traumatized, or, degenerating cartilage, (see, e.g., US 2010/0125341 and US 2010/059495, which are incorporated by reference in their entirety). Within alternative embodiments of the invention, the PEU polymer may be formed on an artificial joint, in order to extend and/or otherwise enhance the effective life of the joint. Representative examples of artificial joints are described in U.S. Pat. Nos. RE 28,895, 7,963,998 and 7,771,485. Within particularly preferred embodiments of the invention, PEU polymers which are placed over the surface of a subject's cartilage (e.g., joint, femoral head of the femoral joint, etc.) or on a medical device, will have a similar coefficient of friction to that of a normal joint, and will help to at least partially restore both normal joint function and eliminate or reduce pain associated with the joint. Particularly preferred PEU polymers for use within the present invention include, for example, PEUT, PEUA, PEUU, PECUT, PECUA, PECUU, PEEUT, PEEUA, PEEUU, PESUT and PESUU.

Compositions and Methods for Application within a PEU-Containing Joint

As described above, the PEU polymers described herein may be applied to the existing joint of a patient (e.g., to the femoral head), in order to preserve cartilage, or to an artificially-created joint. The PEU polymers are designed to be swellable in an aqueous or biological environment, and hence, in certain embodiments of the invention compositions are provided for injection into a joint containing a PEU polymer. Representative examples of suitable compositions include those containing hyaluronic acid, saline, buffered forms of saline, as well as various combinations of these. In addition, within further embodiments the composition may further comprise one or more biologically active agents as noted above.

Within certain additional embodiments of the invention, kits are provided comprising a PEU cap which has been designed for a joint (e.g., a femoral head), and a composition for injection or administration into the joint at the time of surgery, or during subsequent rounds of administration post-surgery. Within certain preferred embodiments the composition for injection or administration into the joint comprises an anesthetic and an anti-inflammatory agent, and optionally, an antibacterial agent. Particularly preferred PEU polymers for use within the present invention include, for example, PEUT, PEUA, PEUU, PECUT, PECUA, PECUU, PEEUT, PEEUA, PEEUU, PESUT and PESUU.

The present invention will be illustrated below with reference to Examples, but is not to be construed as being limited thereto.

### EXAMPLES

### Example 1

### Synthesis and Characterization of Polyether-Urethane

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until the vessel contents are too viscous to continue stifling. Upon obtaining suitable molecular weight, stifling is stopped and the temperature is decreased from 100° C. to room temperature. The final polymer is extracted with deionized water for 24 hours followed by acetone for at least 1 hour to deactivate unreacted isocyanate end groups and to remove any unreacted monomer. The purified polymer is isolated and dried to constant weight at 55° C. in a vacuum oven.

### Example 2

### Synthesis and Characterization of a Polyether-Urea from Polyetherdiamine and Diisocyanate

For an initial charge, poly(tetramethylene ether glycol)diamine (average Mn=1400, 0.0579 moles, 81.06 grams) and poly(propylene glycol-block-ethylene glycol-block-propylene glycol)diamine (average Mn=2000, 0.00493 moles, 9.86 grams) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Tin(II) 2-ethyl hexanoate (0.2 M in dioxane, 5.9243 mL, 0.0011849 moles) is added to the reaction kettle at room temperature and stirred for 30 minutes. Hexamethylene diisocyanate (15.86 grams, 0.0942938 moles) is then added to the solution at room temperature and stirred vigorously until the vessel contents are too viscous to continue stifling. The reaction is kept at room temperature overnight, and on the following day polymer is extracted with deionized water for 24 hours followed by acetone for at least 1 hour to deactivate unreacted isocyanate end groups and to remove the remaining N,N-dimethylacetamide and unreacted monomer. The purified polymer is isolated and dried to constant weight at 55° C. in a vacuum oven.

### Example 3

### Synthesis and Characterization of a Polyether-Urethane-Urea Prepared by Chain Extending with Polyetheramine (2,2′-(Ethylenedioxy)bis(ethylamine))

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours. Upon obtaining suitable conversion, the temperature is decreased to room temperature. At room temperature, the prepolymer is chain extended by the addition of 2,2′-(Ethylenedioxy)bis(ethylamine) (MW=148.20, 0.031431 moles, 4.6581 grams) while stifling vigorously. The contents are stirred at room temperature until the reaction contents are too viscous to continue stifling. The reaction is allowed to stand overnight at room temperature. The final polymer is extracted with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 4

### Synthesis and Characterization of a Polyether-Urethane-Urea Prepared from a Polyether Diamine Copolymer Based on Polytetramethylene Ether Glycol and Polypropylene Glycol, And Chain Extended with a Polyethylene Glycol

For an initial charge, poly(tetramethylene ether-block-propylene ether)diamine (average Mn=1400, 0.0628625 moles, 88.01 grams) is added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until suitable conversion has been attained, and then the temperature is decreased to 25° C. At 25° C. polyethylene glycol (average Mn=1000, 0.031431 moles, 31.43 grams) is added while stifling and allowed to mix for 30 minutes. The temperature is then increased to 80° C. and reacted until the desired molecular weight is obtained at which point the temperature is lowered to room temperature. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 5

### Synthesis and Characterization of a Polyether-Urea Prepared from a Polyether Diamine Copolymer Based on Polytetramethylene Ether Glycol and Polypropylene Glycol, and Chain Extended with Ethylene Diamine

For an initial charge, poly(tetramethylene ether-block-propylene ether)diamine (average Mn=1400, 0.057931 moles, 81.10 grams) is added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 500 mL of anhydrous N,N-dimethylacetamide are added to the reaction kettle through a glass funnel to dissolve the dried reaction components. 2,2′-oxybis-ethanamine (Mn=104, 0.33420 moles, 34.75 grams) and tin(II) 2-ethyl hexanoate (0.2 M in dioxane, 5.9243 mL, 0.0011849 moles) is added to the reaction flask and the contents are stirred gently for at least 30 minutes in order to create a homogeneous solution. Hexamethylene diisocyanate (0.588197 moles, 98.935 grams) is added to the solution drop wise using an addition funnel at room temperature while stirring over a one hour period. The reaction conditions are maintained for 1.25 hours or until suitable conversion has been attained. At 25° C. the prepolymer is chain extended by the addition of ethylene diamine (Mn=60.1, 0.196066 moles, 11.7835 grams) while stirring vigorously. The polymer solution is stirred until it becomes too viscous to continue stifling. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 6

### Synthesis and Characterization of a Polyether-Carbonate-Urethane-Urea that is Chain Extended with an Aliphatic Diamine

For an initial charge, poly(hexamethylene-carbonate)diol (average Mn=1000, 57.931 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until the vessel contents are too thick to continue stirring. Upon obtaining suitable molecular weight, stifling is stopped and the temperature is decreased from 100° C. to room temperature. At 25° C. the prepolymer is chain extended by the addition of ethylene diamine (Mn=60.1, 0.031431 moles, 1.8890 grams) while stifling vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 7

### Synthesis and Characterization of a Polyether-Carbonate-Urethane-Urea that is Chain Extended with a Polyetherdiamine (2,2′-(Ethylenedioxy)bis(ethylamine))

For an initial charge, poly(hexamethylene-carbonate)diol (average Mn=1000, 57.931 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until the vessel contents are too thick to continue stirring. Upon obtaining suitable molecular weight, stifling is stopped and the temperature is decreased from 100° C. to room temperature. At 25° C. the prepolymer is chain extended by the addition of 2,2′-(Ethylenedioxy)bis(ethylamine) (MW=148.20, 0.031431 moles, 4.6581 grams) while stifling vigorously. The polymer solution is stirred until it becomes too viscous to continue stifling. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 8

### Synthesis and Characterization of a Polyether-Urethane-Urea Prepared from a Random Copolymer of Polyethylene Glycol and Polypropylene Glycol and Chain Extended with Hexamethylene Diisocyanate and Ethylene Diamine

For an initial charge, poly(tetramethylene ether)glycol (average Mn=2,900, 168.0 grams, 0.057931 moles) and poly(ethylene glycol-ran-propylene glycol) (average Mn=12000, 59.178 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles)) is added to the solution at room temperature and stirred for 30 minutes. The contents are then heated to 100° C., and tin(II) 2-ethyl hexanoate ((0.2 M in dioxane, 5.9243 mL, 0.0011849 moles)) is added to the reaction to initiate polymerization. The reaction conditions are maintained for 1.25 hours or until suitable conversion has been attained, and then the temperature is decreased to 25° C. At 25° C. the prepolymer is chain extended by the addition of ethylene diamine (Mn=60.1, 0.031431 moles, 1.8890 grams) while stifling vigorously. The polymer solution is stirred until it has become too viscous to continue stifling. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 9

### Synthesis and Characterization of a Polyether-Urethane-Urea Prepared from Aliphatic and Aromatic Diisocyanates

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (0.0471469 moles, 7.9301 grams) and 4,4′-methylenediphenyl diisocyanate (0.0471469 moles, 250.25 g/mol, 11.7985 grams) are added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until suitable conversion has been attained, and then the temperature is decreased to 25° C. At 25° C. the prepolymer is chain extended by the addition of ethylene diamine (Mn=60.1, 0.031431 moles, 1.8890 grams) while stifling vigorously. The polymer solution is stirred until it has become too viscous to continue stifling. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 10

### Synthesis and Characterization of a Polyether-Urethane-Urea Prepared from Aliphatic and Aromatic Diisocyanates and Chain Extended with a Polyether Amine (2,2′-(Ethylenedioxy)bis(ethylamine))

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (0.0471469 moles, 7.9301 grams) and 4,4′-methylenediphenyl diisocyanate (0.0471469 moles, 250.25 g/mol, 11.7985 grams) are added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until suitable conversion has been attained, and then the temperature is decreased to 25° C. At 25° C. the prepolymer is chain extended by the addition of 2,2′-(Ethylenedioxy)bis(ethylamine) (MW=148.20, 0.031431 moles, 4.6581 grams) while stirring vigorously. The polymer solution is stirred until it has become too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 11

### Synthesis and Characterization of a Polyether-Carbonate-Urethane-Urea that is Chain Extended with Ethylene Diamine

For an initial charge, poly(hexamethylene-carbonate)diol (average Mn=1000, 180.0 grams, 0.18 moles) and poly(ethylene glycol-block-propylene glycol-block-ethylene glycol) (average Mn=14,600, 20.0 grams, 0.00137) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (45.76 grams, 0.272055 moles) is added to the solution at room temperature and stirred for 30 minutes. The contents are then heated to 100° C., and tin(II) 2-ethyl hexanoate (0.2 M in dioxane, 5.9243 mL, 0.0011849 moles) is added to the reaction to initiate polymerization. The reaction conditions are maintained for 1.25 hours or until the vessel contents are too thick to continue stifling. Upon obtaining suitable molecular weight, stifling is stopped and the temperature is decreased from 100° C. to room temperature. At 25° C. the prepolymer is chain extended by the addition of ethylene diamine (0.090685 moles, 5.45 grams) while stirring vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 12

### Synthesis of a Polyether-Siloxane-Urethane-Urea that is Chain Extended with an Aliphatic Diamine

For an initial charge, poly(dimethylsiloxane), bis(hydroxyalkyl) terminated (average Mn=5,600, 70.0 grams, 0.0125 moles) and poly(ethylene glycol-block-propylene glycol-block-ethylene glycol) (average Mn=14,600, 30.0 grams, 0.00205479 moles) are added to a 1000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 500 mL of anhydrous dimethylformamide are added to the reaction kettle through a glass funnel to dissolve the dried reaction components. The contents are stirred gently for at least 30 minutes in order to create a homogeneous solution. Hexamethylene diisocyanate (3.668 grams, 0.021832 moles) is added to the solution at room temperature and stirred for 30 minutes. The contents are then heated to 100° C., and tin(II) 2-ethyl hexanoate (0.2 M in dioxane, 1.455 mL, 0.000291 moles) is added to the reaction to initiate polymerization. The reaction conditions are maintained for 1.25 hours or until the vessel contents are too thick to continue stifling. Upon obtaining suitable molecular weight, stifling is stopped and the temperature is decreased from 100° C. to room temperature. At 25° C. the prepolymer is chain extended by the addition of ethylene diamine (Mn=60.1, 0.007277 moles, 0.4374 grams) while stifling vigorously. The polymer solution is stirred until it becomes too viscous to continue stifling. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

The present disclosure provides the following numbered embodiments, which are exemplary of the embodiments provided herein.
1) A polymer composition which is the reaction product of a pre-polymer and a diamine, where the pre-polymer is the reaction product of a diisocyanate and a polyetherdiol.
2) The polymer of embodiment 1 wherein the polyetherdiol comprises at least one type of oxyalkylene sequence selected from the group consisting of oxyethylene, oxypropylene, oxytrimethylene and oxytetramethylene sequences.
3) The polymer of embodiments 1-2 wherein the polyetherdiol is a blend of polyetherdiols.
4) The polymer of embodiments 1-2 wherein the polyether diol is not a blend of polyetherdiols.
5) The polymer of embodiments 1-4 wherein the polyetherdiol is a random copolymer of two or more oxyalkylene sequences.
6) The polymer of embodiments 1-4 wherein the polyetherdiol is a block copolymer of two or more oxyalkylene sequences.
7) The polymer of embodiments 1-6 wherein the diamine is an aliphatic diamine.
8) The polymer of embodiments 1-6 wherein the diamine is a polyether diamine.
9) The polymer of embodiments 1-6 wherein the diamine is a blend of diamines.
10) The polymer of embodiments 1-6 wherein the diamine is a blend of aliphatic diamine and polyether diamine.
11) A polymer composition which is the reaction product of a pre-polymer and a diol, where the pre-polymer is the reaction product of a diisocyanate and a polyetherdiamine.
12) The polymer of embodiment 11 wherein the polyetherdiamine comprises at least one type of oxyalkylene sequence selected from the group consisting of oxyethylene, oxypropylene, oxytrimethylene and oxytetramethylene sequences.
13) The polymer of embodiments 11 or 12 wherein the polyetherdiamine is a blend of polyetherdiamines.
14) The polymer of embodiments 11 or 12 wherein the polyetherdiamine is not a blend of polyetherdiamines.
15) The polymer of embodiments 11-14 wherein the polyetherdiamine is a random copolymer of two or more oxyalkylene sequences.
16) The polymer of embodiments 11-14 wherein the polyetherdiamine is a block copolymer of two or more oxyalkylene sequences.
17) The polymer of embodiments 11-16 wherein the diol is an aliphatic diol.
18) The polymer of embodiments 11-16 wherein the diol is an aromatic diol.
19) The polymer of embodiments 11-16 wherein the diol is a polyether diol.
20) The polymer of embodiments 11-16 wherein the diol is a blend of diols.
21) The polymer of embodiments 11-16 wherein the diol is a blend of aliphatic diol and polyetherdiol.
22) The polymer of embodiments 1-21 wherein the diisocyanate is an aliphatic diisocyanate and the reactants do not include an aromatic diisocyanatae to form a polyether urea urethane.
23) The polymer of embodiments 1-20 wherein the diisocyanate is a mixture of aliphatic diisocyanates and the reactants do not include an aromatic diisocyanate to form a polyether urea urethane.
24) The polymer of embodiments 1-20 wherein the diisocyanate is an aromatic diisocyanate and the reactants do not include an aliphatic diisocyanate to form a polyether urea urethane.
25) The polymer of embodiments 1-20 wherein the diisocyanate is a mixture of aromatic diisocyanates and the reactants do not include an aliphatic diisocyanate to form a polyether urea urethane.
26) The polymer of embodiments 1-20 wherein the diisocyanate is a mixture of aromatic diisocyanate and aliphatic diisocyanate to form a polyether urea urethane.
27) A polymer composition which is the reaction product of a diisocyanate and a diol.
28) The polymer of embodiment 27 wherein the diol is a polyether diol.
29) The polymer of embodiment 28 wherein the polyether diol comprises at least one type of oxyalkylene sequence selected from the group consisting of oxyethylene, oxypropylene, oxytrimethylene and oxytetramethylene sequences.
30) The polymer of embodiments 27 or 28 wherein the polyetherdiol is a blend of polyetherdiols.
31) The polymer of embodiments 27 or 28 wherein the polyether diol is not a blend of polyetherdiols.
32) The polymer of any of embodiments 28-31 wherein the polyetherdiol is a random copolymer of two or more oxyalkylene sequences.
33) The polymer of any of embodiments 28-31 wherein the polyetherdiol is a block copolymer of two or more oxyalkylene sequences.
34) The polymer of any of embodiments 27-33 wherein the diisocyanate is an aliphatic diisocyanate and the reactants do not include an aromatic diisocyanatae.
35) The polymer of any of embodiments 27-33 wherein the diisocyanate is a mixture of aliphatic diisocyanates and the reactants do not include an aromatic diisocyanate.
36) The polymer of any of embodiments 27-33 wherein the diisocyanate is an aromatic diisocyanate and the reactants do not include an aliphatic diisocyanate.
37) The polymer of any of embodiments 27-33 wherein the diisocyanate is a mixture of aromatic diisocyanates and the reactants do not include an aliphatic diisocyanate.
38) The polymer of any of embodiments 27-33 wherein the diisocyanate is a mixture of aromatic diisocyanate and aliphatic diisocyanate.
39) The polymer of any of embodiments 27-38 wherein diisocyanate and diol are the only reactants.
40) The polymer of any of embodiments 28-39 wherein the molar ratio of diisocyanate to polyether diol is in the range of 0.95 to 1.05.
41) A polymer composition which is the reaction product of a diisocyanate and a diamine.
42) The polymer of embodiment 41 wherein the diamine is a polyether diamine.
43) The polymer of embodiment 42 wherein the polyether diamine comprises at least one type of oxyalkylene sequence selected from the group consisting of oxyethylene, oxypropylene, oxytrimethylene and oxytetramethylene sequences.
44) The polymer of embodiments 41 or 42 wherein the polyetherdiamine is a blend of polyetherdiamines.
45) The polymer of embodiments 41 or 42 wherein the polyether diamine is not a blend of polyetherdiamines.
46) The polymer of any of embodiments 42-45 wherein the polyetherdiamine is a random copolymer of two or more oxyalkylene sequences.
47) The polymer of any of embodiments 42-45 wherein the polyetherdiamine is a block copolymer of two or more oxyalkylene sequences.
48) The polymer of any of embodiments 41-47 wherein the diisocyanate is an aliphatic diisocyanate and the reactants do not include an aromatic diisocyanatae.
49) The polymer of any of embodiments 41-47 wherein the diisocyanate is a mixture of aliphatic diisocyanates and the reactants do not include an aromatic diisocyanate.
50) The polymer of any of embodiments 41-47 wherein the diisocyanate is an aromatic diisocyanate and the reactants do not include an aliphatic diisocyanate.
51) The polymer of any of embodiments 41-47 wherein the diisocyanate is a mixture of aromatic diisocyanates and the reactants do not include an aliphatic diisocyanate.
52) The polymer of any of embodiments 41-47 wherein the diisocyanate is a mixture of aromatic diisocyanate and aliphatic diisocyanate.
53) The polymer of any of embodiments 41-52 wherein diisocyanate and diamine are the only reactants.
54) The polymer of any of embodiments 42-53 wherein the molar ratio of diisocyanate to polyether diamine is in the range of 0.95 to 1.05.
55) A polymer composition which is the reaction product of a diisocyanate and either (a) a mixture comprising polyether diol and polycarbonate diol or (b) a polyether polycarbonate diol.
56) The polymer of embodiment 55 wherein the polyetherdiol comprises at least one type of oxyalkylene sequence selected from the group consisting of oxyethylene, oxypropylene, oxytrimethylene and oxytetramethylene sequences.
57) The polymer of embodiments 55 or 56 wherein the polyetherdiol is a blend of polyetherdiols.
58) The polymer of embodiments 55 or 56 wherein the polyether diol is not a blend of polyetherdiols.
59) The polymer of any one of embodiments 55-58 wherein the polyetherdiol is a random copolymer of two or more oxyalkylene sequences.
60) The polymer of any one of embodiments 55-58 wherein the polyetherdiol is a block copolymer of two or more oxyalkylene sequences.
61) The polymer of any one of embodiments 55-60 wherein the polycarbonate diol is poly(hexamethylene carbonate)diol.
62) The polymer of any one of embodiments 55-60 wherein the polycarbonate diol is poly(ethylene-carbonate)diol.
63) The polymer of any one of embodiments 55-60 wherein the polycarbonate diol is the reaction product of trimethylene carbonate and a diol.
64) The polymer of any one of embodiments 55-63 wherein the diisocyanate is an aliphatic diisocyanate and the reactants do not include an aromatic diisocyanatae.
65) The polymer of any one of embodiments 55-63 wherein the diisocyanate is a mixture of aliphatic diisocyanates and the reactants do not include an aromatic diisocyanate.
66) The polymer of any one of embodiments 55-63 wherein the diisocyanate is an aromatic diisocyanate and the reactants do not include an aliphatic diisocyanate.
67) The polymer of any one of embodiments 55-63 wherein the diisocyanate is a mixture of aromatic diisocyanates and the reactants do not include an aliphatic diisocyanate.
68) The polymer of any one of embodiments 55-63 wherein the diisocyanate is a mixture of aromatic diisocyanate and aliphatic diisocyanate.
69) The polymer of any one of embodiments 55-68 which is further chain extended by reaction with a diol.
70) A polymer composition which is the reaction product of a diamine and a pre-polymer, where the pre-polymer is the reaction product of a diisocyanate and either (a) a mixture comprising polyether diol and polycarbonate diol or (b) a polyether polycarbonate diol.
71) The polymer of embodiment 70 wherein the polyetherdiol comprises at least one type of oxyalkylene sequence selected from the group consisting of oxyethylene, oxypropylene, oxytrimethylene and oxytetramethylene sequences.
72) The polymer of embodiments 70 or 71 wherein the polyetherdiol is a blend of polyetherdiols.
73) The polymer of embodiments 70 or 71 wherein the polyether diol is not a blend of polyetherdiols.
74) The polymer of any one of embodiments 70-73 wherein the polyetherdiol is a random copolymer of two or more oxyalkylene sequences.
75) The polymer of any one of embodiments 70-73 wherein the polyetherdiol is a block copolymer of two or more oxyalkylene sequences.
76) The polymer of any one of embodiments 70-75 wherein the polycarbonate diol is poly(hexamethylene carbonate)diol.
77) The polymer of any one of embodiments 70-75 wherein the polycarbonate diol is poly(ethylene-carbonate)diol.
78) The polymer of any one of embodiments 70-75 wherein the polycarbonate diol is the reaction product of trimethylene carbonate and a diol.
79) The polymer of any one of embodiments 70-78 wherein the diisocyanate is an aliphatic diisocyanate and the reactants do not include an aromatic diisocyanatae.
80) The polymer of any one of embodiments 70-78 wherein the diisocyanate is a mixture of aliphatic diisocyanates and the reactants do not include an aromatic diisocyanate.
81) The polymer of any one of embodiments 70-78 wherein the diisocyanate is an aromatic diisocyanate and the reactants do not include an aliphatic diisocyanate.
82) The polymer of any one of embodiments 70-78 wherein the diisocyanate is a mixture of aromatic diisocyanates and the reactants do not include an aliphatic diisocyanate.
83) The polymer of any one of embodiments 70-78 wherein the diisocyanate is a mixture of aromatic diisocyanate and aliphatic diisocyanate.
84) The polymer of any one of embodiments 70-83 wherein the diamine is an aliphatic diamine.
85) The polymer of any one of embodiments 70-83 wherein the diamine is a polyether diamine.
86) The polymer of any one of embodiments 70-83 wherein the diamine is a blend of diamines.
87) The polymer of any one of embodiments 70-83 wherein the diamine is a blend of aliphatic diamine and polyether diamine.
88) A polymer composition which is the reaction product of a diisocyanate and either (a) a mixture comprising polyether diol and polyester diol or (b) a polyether polyester diol.
89) The polymer of embodiment 88 wherein the polyether diol comprises at least one type of oxyalkylene sequence selected from the group consisting of oxyethylene, oxypropylene, oxytrimethylene and oxytetramethylene sequences.
90) The polymer of embodiments 88 or 89 wherein the polyether diol is a blend of polyetherdiols.
91) The polymer of embodiments 88 or 89 wherein the polyether diol is not a blend of polyetherdiols.
92) The polymer of any one of embodiments 88-91 wherein the polyether diol is a random copolymer of two or more oxyalkylene sequences.
93) The polymer of any one of embodiments 88-91 wherein the polyether diol is a block copolymer of two or more oxyalkylene sequences.
94) The polymer of any one of embodiments 88-693 wherein the diisocyanate is an aliphatic diisocyanate and the reactants do not include an aromatic diisocyanatae.
95) The polymer of any one of embodiments 88-93 wherein the diisocyanate is a mixture of aliphatic diisocyanates and the reactants do not include an aromatic diisocyanate.
96) The polymer of any one of embodiments 88-93 wherein the diisocyanate is an aromatic diisocyanate and the reactants do not include an aliphatic diisocyanate.
97) The polymer of any one of embodiments 88-93 wherein the diisocyanate is a mixture of aromatic diisocyanates and the reactants do not include an aliphatic diisocyanate.
98) The polymer of any one of embodiments 88-93 wherein the diisocyanate is a mixture of aromatic diisocyanate and aliphatic diisocyanate.
99) A polymer composition which is the reaction product of a diamine and a pre-polymer, where the pre-polymer is the reaction product of a diisocyanate and either (a) a mixture comprising polyether diol and polyester diol or (b) a polyether polyester diol.
100) The polymer of embodiment 99 wherein the polyetherdiol comprises at least one type of oxyalkylene sequence selected from the group consisting of oxyethylene, oxypropylene, oxytrimethylene and oxytetramethylene sequences.
101) The polymer of embodiments 99 or 100 wherein the polyetherdiol is a blend of polyetherdiols.
102) The polymer of embodiments 99 or 100 wherein the polyether diol is not a blend of polyetherdiols.
103) The polymer of any one of embodiments 99-102 wherein the polyetherdiol is a random copolymer of two or more oxyalkylene sequences.
104) The polymer of any one of embodiments 99-102 wherein the polyetherdiol is a block copolymer of two or more oxyalkylene sequences.
105) The polymer of any one of embodiments 99-104 wherein the diisocyanate is an aliphatic diisocyanate and the reactants do not include an aromatic diisocyanatae.
106) The polymer of any one of embodiments 99-104 wherein the diisocyanate is a mixture of aliphatic diisocyanates and the reactants do not include an aromatic diisocyanate.
107) The polymer of any one of embodiments 99-104 wherein the diisocyanate is an aromatic diisocyanate and the reactants do not include an aliphatic diisocyanate.
108) The polymer of any one of embodiments 99-104 wherein the diisocyanate is a mixture of aromatic diisocyanates and the reactants do not include an aliphatic diisocyanate.
109) The polymer of any one of embodiments 99-104 wherein the diisocyanate is a mixture of aromatic diisocyanate and aliphatic diisocyanate.
110) The polymer of any one of embodiments 99-109 wherein the diamine is an aliphatic diamine.
111) The polymer of any one of embodiments 99-109 wherein the diamine is a polyether diamine.
112) The polymer of any one of embodiments 99-109 wherein the diamine is a blend of diamines.
113) The polymer of any one of embodiments 99-109 wherein the diamine is a blend of aliphatic diamine and polyether diamine.
114) The polymer any of one of embodiments 1-113 which is bio-stable.
115) The polymer of any one of embodiments 1-114 which absorbs at least 50% of its weight in water when immersed in 1% aqueous methyl cellulose at 37° C. for 16 hours.
116) The polymer of any one of embodiments 1-115 which has a COF of 0.001 to 0.15.
117) The polymer of any one of embodiments 1-116 which has an intrinsic viscosity of 3-8 dl/g.

What is claimed is:

1. A polymer composition which is the reaction product of a pre-polymer and a diamine, where the pre-polymer is the reaction product of a diisocyanate and a polyetherdiol.

2. The polymer of claim 1 wherein the polyetherdiol comprises at least one type of oxyalkylene sequence selected from the group consisting of oxyethylene, oxypropylene, oxytrimethylene and oxytetramethylene sequences.

3. The polymer of claim 1 wherein the polyetherdiol is a blend of polyetherdiols.

4. The polymer of claim 1 wherein the polyetherdiol comprises a block copolymer of two or more oxyalkylene sequences.

5. The polymer of claim 1 wherein the diamine is an aliphatic diamine.

6. The polymer of claim 1 wherein the diisocyanate is an aliphatic diisocyanate.

7. The polymer claim 1 which is bio-stable.

8. The polymer of claim 1 which absorbs at least 50% of its weight in water when immersed in 1% aqueous methyl cellulose at 37° C. for 16 hours.

9. The polymer of claim 1 which has a COF of 0.001 to 0.15.

10. The polymer of claim 1 which has an intrinsic viscosity of 3-8 dl/g.

11. A polymer composition which is the reaction product of a pre-polymer and a polyetherdiol, where the pre-polymer is the reaction product of a diisocyanate and a diamine.

12. The polymer of claim 11 wherein the polyetherdiol comprises at least one type of oxyalkylene sequence selected from the group consisting of oxyethylene, oxypropylene, oxytrimethylene and oxytetramethylene sequences.

13. The polymer of claim 11 wherein the polyetherdiol is a blend of polyetherdiols.

14. The polymer of claim 11 wherein the polyetherdiol comprises a block copolymer of two or more oxyalkylene sequences.

15. The polymer of claim 11 wherein the diamine is an aliphatic diamine.

16. The polymer of claim 11 wherein the diisocyanate is an aliphatic diisocyanate.

17. The polymer claim 11 which is bio-stable.

18. The polymer of claim 11 which absorbs at least 50% of its weight in water when immersed in 1% aqueous methyl cellulose at 37° C. for 16 hours.

19. The polymer of claim 11 which has a COF of 0.001 to 0.15.

20. The polymer of claim 11 which has an intrinsic viscosity of 3-8 dl/g.
